SEQUENCE LISTING (2 sequences)

SEQ ID NO: 1 - DNA, 1517 bp, Human papillomavirus type 16
  misc_feature (1)..(1517): Variant HPV16/31 LI protein nucleotide sequence
atgagcctgtggctgcccagcgaggccaccgtgtacctgccccccgtgcccgtgagcaaggtgtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccaccgacctgtacatcaagggcagcggcagcaccgccaccctggccaacagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtga

SEQ ID NO: 2 - DNA, 1488 bp, Bovine papillomavirus type 1
  misc_feature (1)..(1488): BPV1 LI nucleotide sequence
atggccctctggcagcaggggcagaaactctacctgccacccacacccgtgtcaaaagtcctgtgttccgagacatacgtccagcggaagtcaatcttctaccacgccgagaccgaaaggctcctcaccatcggccacccctactaccccgtcagcattggcgctaagaccgtgcccaaagtctccgccaaccaataccgcgtgttcaagatccagctgcccgacccaaaccagttcgccctgcccgatcgcaccgtgcataacccctccaaggaaagactcgtctgggccgtgatcggcgtccaagtctcacggggccaacccctgggcggcaccgtgaccggccatccaaccttcaacgccctcctggacgccgagaacgtcaaccggaaagtcacaacacaaaccaccgacgatcgcaagcagaccgggctggacgccaaacagcagcaaatcctcctcctggggtgcacacccgctgagggcgagtactggaccaccgctcggccctgcgtgaccgacaggctggagaacggggcttgtccccccctggagctgaagaataagcatatcgaggacggcgacatgatggagatcggcttcggcgccgctaacttcaaggagatcaacgcctccaagagcgacctgcccctggatatccagaacgaaatttgtctctatcccgattatctgaagatggccgaagatgccgccggcaactcaatgtttttcttcgcccgcaaggagcaagtctacgtgcggcatatttggacacggggcgggagcgaaaaggaggctcccacaaccgacttctacctgaaaaacaacaagggcgacgctacactgaagatcccatccgtccacttcggctccccatccgggagcctcgtcagcaccgacaaccagatcttcaacagaccatattggctgtttagggctcaagggatgaataacggcatcgcttggaacaacctgctcttcctgaccgtcggcgataacaccaggggcaccaacctgacaatctccgtggctagcgacggcacacccctgaccgaatacgactcaagcaagtttaacgtgtatcaccggcacatggaggagtacaaactggctttcatcctggaactgtgtagcgtcgagattaccgcccagaccgtcagccacctccagggcctgatgccaagcgtcctggagaactgggagatcggcgtccaaccaccaacaagcagcatcctggaagatacatacagatacatcgaaagccccgccaccaagtgcgcctcaaacgtgatccccgccaaggaggatccctacgccggcttcaaattctggaatatcgacctgaaggagaaactgagcctcgatctggaccagttcccactcggccggcggttcctggcccaacagggcgctggctgcagcaccgtccggaagaggcggatctcacaaaagaccagttccaaacccgccaagaagaagaagaagtag

(19) United States
(12) Patent Application Publication
(10) Pub. No.: US 2019/0070296 A1
(43) Pub. Date: Mar. 7, 2019

(54) SONODYNAMIC THERAPY

(71) Applicant: LI-COR, INC., Lincoln, NE (US)

(72) Inventors: Han-Wei WANG, Lincoln, NE (US); Lyle R. MIDDENDORF, Lincoln, NE (US)

(73) Assignee: LI-COR, INC., Lincoln, NE (US)

(21) Appl. No.: 16/103,680

(22) Filed: Aug. 14, 2018

Related U.S. Application Data
(63) Continuation of application No. PCT/US18/45763, filed on Aug. 8, 2018.
(60) Provisional application No. 62/547,267, filed on Aug. 18, 2017.

(51) Int. Cl. A61K 41/00; A61K 47/69

(52) U.S. Cl. CPC A61K 41/0033; A61P 35/00; A61K 47/6925

(57) ABSTRACT

The present disclosure provides methods for treating diseased cells in a subject using sonodynamic therapy (SDT), comprising: administering to the subject a sonosensitizer composition comprising IRDye® 700DX, wherein the sonosensitizer composition associates with the diseased cell; and thereafter applying an ultrasonic wave to the diseased cell.

### CROSS-REFERENCES TO RELATED APPLICATIONS

This application is a continuation of PCT Application No. PCT/US18/45763, filed Aug. 8, 2018 which claims priority to U.S. Provisional Patent Application No. 62/547,267, filed Aug. 18, 2017, the disclosure of which is hereby incorporated by reference in its entirety for all purposes.

### REFERENCE TO A SEQUENCE LISTING

The sequence listing written in file SEQUENCELISTING_1090039.TXT created on Aug. 14, 2018, 4,598 bytes, machine format IBM-PC, MS-Windows operating system, is hereby incorporated by reference in its entirety for all purposes.

### BACKGROUND OF THE INVENTION

Cancer has become the leading cause of death worldwide and has attracted a lot of attention in clinical research. To treat the prevalent disease, four primary approaches including surgery, chemotherapy, radiotherapy, and immunotherapy can be applied to patients. However, each treatment has its own limitations that leads to the combination uses of treatments and development efforts of new treatment methods.

For instance, cancer surgery can have difficulty on clearing all cancer cells around the primary tumor site and is not used to cure the metastasized tumor. Chemotherapy and radiotherapy can be effective on eliminating cancer cells, but normal tissues can be damaged at the same time. In addition, the main obstacle on these therapies is the development of drug tolerance during the period of chemotherapy and radiotherapy.

Immunotherapy is often effective treatment on cancers, but is costly and can cause fatal immune reaction. Therefore, other research and applications of different types of cancer therapy are important and helpful in the progress of curing cancer.

Sonodynamic therapy (SDT) is an emerging option for the minimally invasive treatment of solid cancerous tumors. In general, SDT combines three working parts: a sensitizing drug or sensitizer, an activation power source, and molecular oxygen. However, different from photodynamic therapy, which technique uses light, SDT uses a sonic or an ultrasound source for activation. The advantage is that ultrasound waves can penetrate much deeper into human tissue than light photons due to greatly reduced energy attenuation, thus leading to a treatment at a deep area of the human body in a minimally invasive way.

In view of the foregoing, there is a need in the art for new sonosensitizers and therapeutic methods of using them. New sonosensitizers are needed that can be used to treat a wide variety of tumors at deeper and less accessible sites than available today. The current disclosure satisfies these and other needs.

### BRIEF SUMMARY OF THE INVENTION

This disclosure provides methods of using IRDye® 700DX with an ultrasonic power source to generate cytotoxic effect for therapeutic treatment. In certain aspects, the compositions comprise IRDye® 700DX and optionally a carrier moiety such as nanoparticles and/or targeting moieties such as antibodies to enhance and amplify the sonodynamic effect. In certain aspects, the disclosure relates to a method of sonodynamic therapy, which comprises administering to a subject a therapeutically effective amount of a composition of the disclosure, followed by local ultrasound.

As such, in one embodiment, the present disclosure provides a method for treating a diseased cell in a subject using sonodynamic therapy (SDT), the method comprising:

administering to the subject a sonosensitizer composition comprising IRDye® 700DX, wherein the sonosensitizer composition associates with the diseased cell; and

applying an ultrasonic wave to the diseased cell.

In certain instances, the diseased cell is a cancer cell such as a solid tumor. In certain aspects, the SDT methods described herein are used to treat symptoms or improve conditions associated with various disease states, including cancer. In one aspect, the methods include sonodynamic therapy and together with chemotherapeutic drugs. In certain aspects, the sonosensitizing composition is specifically absorbed in tumor cells and produces cytotoxic moieties such as singlet oxygen.

In certain other instances, the present disclosure provides a kit comprising a sonosensitizer composition comprising IRDye® 700DX. In certain aspects, the sonosensitizer composition is liquid or a solid such as a powder, which can be reconstituted into a liquid. The kit optionally comprises at least one syringe and/or one needle. And yet another aspect, the disclosure provides a syringe prefilled with a liquid sonosensitizer composition.

These and other aspects, objects and embodiments will become more apparent when read with the detailed description and figures which follow.

### BRIEF DESCRIPTION OF THE DRAWINGS

FIG. 1A-1C illustrate various sonosensitizer composition embodiments comprising IRDye® 700DX of the present disclosure.

FIG. 2 shows an exemplary embodiment of a sonodynamic composition comprising IRDYE® 700DX in accordance with an embodiment of the present disclosure.

FIG. 3 shows the results of sonodynamic experiment using methods comprising IR DYE® 700DX in accordance with an embodiment of the present disclosure.

FIG. 4 shows the results of sonodynamic experiment using methods comprising IR DYE® 700DX in accordance with an embodiment of the present disclosure.

FIG. 5 shows the generation of singlet oxygen using methods comprising IR DYE® 700DX in accordance with an embodiment of the present disclosure.

### DETAILED DESCRIPTION OF THE INVENTION

### I. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “sonodynamic therapy” or “SDT” includes a non-surgical treatment of cells, tissues or organs with both a sonosensitizing agent and ultrasound to generate cytotoxic reactive oxygen species in situ, which can inactivate cells. The sonosensitizing agent is excited using ultrasound and not an external light source. The oxygen species can be oxygen radicals and/or singlet oxygen which cause oxidative destruction of tissues.

The term “cytotoxicity” includes the death of a cell or the process thereof due to exposing the cell to a toxin.

The term “cytotoxic agent” includes a substance that inhibits or prevents a cellular function and/or causes cell death or destruction.

The term “cancer” includes the physiological condition in mammals that is typically characterized by unregulated cell growth. Non-limiting examples of cancer include carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “solid tumor” refers to an abnormal mass of cells that are either benign or malignant and usually do not contain cysts. Non-limiting examples of a solid tumor include glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

The term “phthalocyanine dye” includes a silicon phthalocyanine dye that is useful for conjugating to a nanocarrier or targeting agent. Non-limiting examples of a phthalocyanine dye, such as IRDye® 700DX are described in, e.g., U.S. Pat. No. 7,005,518, the disclosure of which is herein incorporated by reference in its entirety for all purposes.

The term “IRDye® 700DX ” or “IR700” is a phthalocyanine dye, which may be optionally conjugated to a nanocarrier and/or targeting agent, which is a therapeutic effective agent. The IRDye® 700DX dye may have an NHS ester linkage to allow for conjugation to a nanocarrier and/or targeting agent. In some instances, the nanocarrier and/or targeting agent has a primary amine (e.g., an amino group) wherein the NHS ester of 700DX and the amino group of the a nanocarrier and/or targeting agent react to form an amide bond, linking the a nanocarrier and/or targeting agent to 700DX to form the therapeutic effective agent. The NHS ester IRDye° 700DX with a linking group has the following structure:

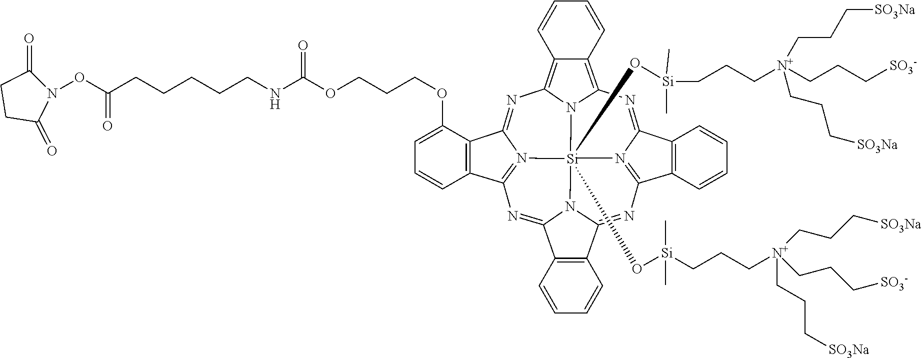

The dye is commercially available from LI-COR (Lincoln, Nebr.). Amino-reactive IRDye® 700DX is a relatively hydrophilic dye and can be optionally covalently conjugated with a nanocarrier using the NHS ester of IRDye® 700DX. Other variations of IRDye 700DX are disclosed in U.S. Pat. No. 7,005,518 (incorporated herein by reference), and those too are useful in the present disclosure. A carboxylate derivative having a linking group has the following name and structure, silicate(5-), bis[N-[3-[(hydroxy-.kappa.O)dimethylsilyl]propyl]-3-sulfo-N,N-bis(3-sulfopropyl)-1-propanaminiumato(4-)][6-[[[3-[(29H,31H-phthalocyanin-yl-kappa.N29,.kappa.N30,.kappa.N31,.kappa.N32)oxy]propoxy]carbonyl]amino]hexanoato(3-)]-, sodium (1:5) CAS Registry Number: [1623074-46-3]:

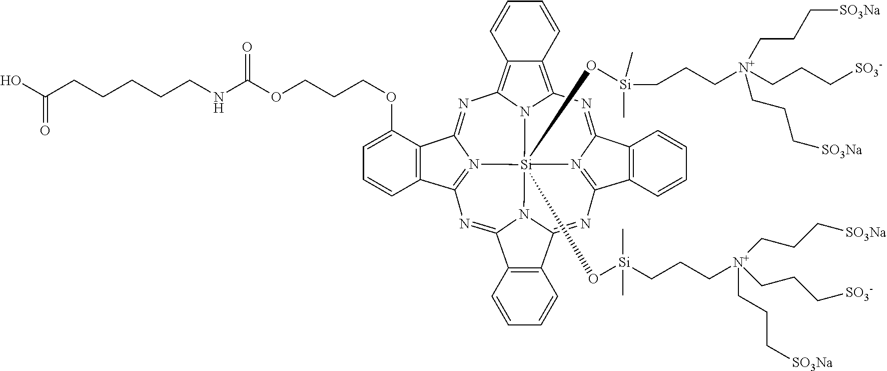

The term “conjugated,” “coupled” or “labeled” refers to linking of a first chemical moiety to a second chemical moeity by means of a suitable crosslinker capable of covalently binding the moiety to the protein.

The term “linking group” includes a moiety on the compound that is capable of chemically reacting with a functional group on a different material (e.g., a nanocarrier and/or targeting agent) to form a linkage, such as a covalent linkage. See, e.g., R. Haughland, The Handbook—A Guide to Fluorescent Probes and Labeling Technologies, 9^{th} Edition, Molecular Probes, Inc. (1992). Typically, the linking group is an electrophile or nucleophile that can form a covalent linkage through exposure to the corresponding functional group that is a nucleophile or electrophile, respectively. Alternatively, the linking group is a photoactivatable group, and becomes chemically reactive only after illumination with light of an appropriate wavelength. Typically, the conjugation reaction between the dye bearing the linking group and the material to be conjugated with the dye “the carrier” results in one or more atoms of the linking group being incorporated into a new linkage attaching the dye to the a nanocarrier and/or targeting agent to form the therapeutic agent.

The term “linker” includes the atoms joining the dye (e.g., IRDye® 700DX) to a carrier and/or targeting agent.

The term “small molecule” includes compositions of matter that are typically less than 1000 g/mol in molecular weight and include inhibitors, carbohydrates, drugs, dyes or cytotoxic agents.

The terms “treat” and “treatment” refer to both therapeutic treatment and prophylactic or preventative measures, wherein the object is to prevent or slow down (lessen) an undesired physiological change or disorder, such as the growth, development or spread of cancer. For example, beneficial or desired clinical results include, but are not limited to, alleviation of symptoms, diminishment of extent of disease, stabilized (i.e., not worsening) state of disease, delay or slowing of disease progression, amelioration of the disease state, and remission (whether partial or total), whether detectable or undetectable. “Treatment” can also mean prolonging survival as compared to expected survival if not receiving treatment. Those in need of treatment include those already with the condition or disorder as well as those prone to have or suspected to have the condition or disorder or those in which the condition or disorder is to be prevented.

The term “therapeutically effective amount” refers to an amount of a composition that alone, or together with an additional therapeutic agent(s) (such as a chemotherapeutic agent) is sufficient to achieve a desired effect in a subject, or in a cell, being treated with the composition. The effective amount of the therapeutic agent or composition (such as an IRDye® 700DX) can be dependent on several factors, including, but not limited to, the subject or cells being treated, the particular therapeutic agent, and the manner of administration of the therapeutic composition. For example, a therapeutically effective amount or concentration is sufficient to prevent advancement (such as metastasis), delay progression, or to cause regression of a disease, or which is capable of reducing symptoms caused by the disease, such as cancer. For instance, a therapeutically effective amount or concentration is sufficient to increase the survival time of a patient with a circulating tumor cell.

The term “subject,” “patient,” or “individual” typically refers to humans, but also to other animals including, e.g., other primates, rodents, canines, felines, equines, ovines, porcines, and the like.

### II. Detailed Descriptions of Embodiments

The present disclosure provides methods for treating a diseased cell in a subject using IRDye® 700DX. In one aspect, a composition comprising a sonosensitizer comprising IRDye® 700DX is administered to a subject. Ultrasound is then used as an activation power source together with the sonosensitizing composition, to generate reactive oxygen species (e.g., radicals or singlet oxygen) to kill or eliminate the cells. The reactive oxygen species are cytotoxic.

As such, in one embodiment, the present disclosure provides a method for treating a diseased cell in a subject using sonodynamic therapy (SDT), the method comprising: administering to the subject a sonosensitizer composition comprising IRDye® 700DX, wherein the sonosensitizer composition associates with the diseased cell; and applying an ultrasonic wave to the diseased cell.

Sonodynamic therapy in the methods described herein include non-surgical treatment of cells, tissues or organs with both a sonosensitizing agent and ultrasound to generate cytotoxic reactive oxygen species in situ, which can inactivate cells. Ultrasound has the capability of treating regions deep in the body (>1 cm) where light would either be blocked, or require more invasive delivery methods. In certain aspects, ultrasound can also provide conformal dosage of energy, and thus induce apoptosis and/or necrosis throughout the entire tumor or tissue. Furthermore, toxicity can be induced in a precise location while minimizing harm to other areas of the body. The methods described herein can be performed in vitro or in vivo. In certain aspects, no external light irradiation is used as IRDye® 700DX is used only as a sonosensitzer agent.

A. Sonosensitizer Compositions

The present disclosure provides sonosensitizer compositions comprising IRDye® 700DX. The term “IRDye® 700DX ” or “IR700” is a phthalocyanine dye, which can be used directly. In other aspects, IRDye® 700DX can comprise a carrier, such as a nanoparticle and/or a targeting agent.

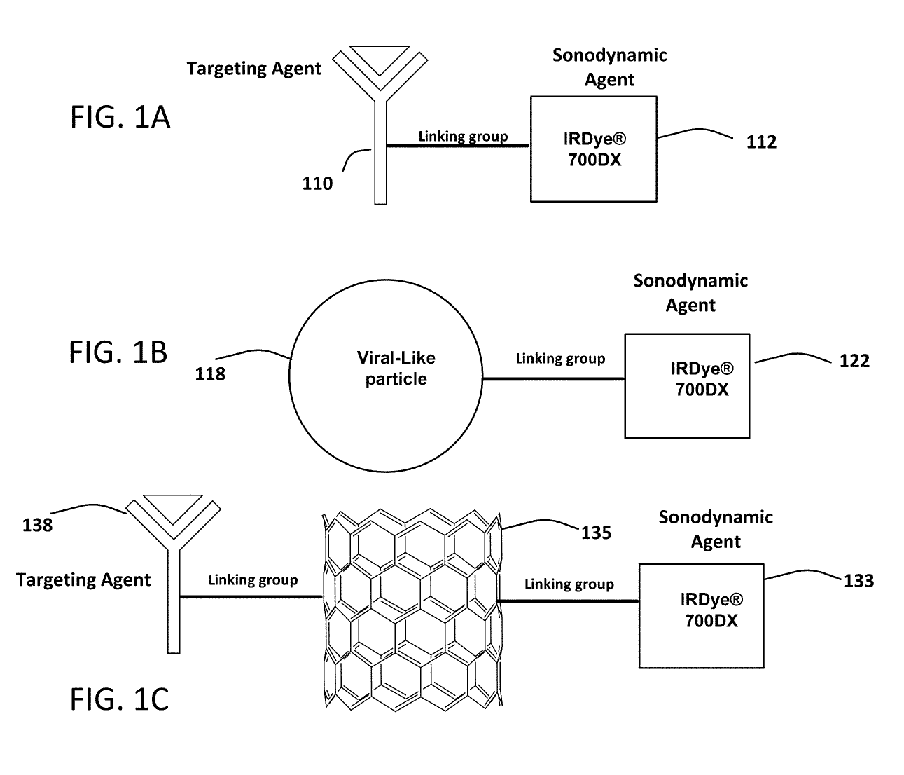

FIG. 1A-1C illustrate various sonosensitizer composition embodiments comprising IRDye® 700DX of the present disclosure. FIG. 1A shows one or more sonosensitizer molecules 112 linked to targeting agent 110 directly with no carrier intermediary. In this non-limiting example, one or more IRDye® 700 molecules 112 is conjugated to one or more antibody molecules 110 by a linking group. The targeting agent 110 can be a small molecule.

Turning now to FIG. 1B, one or more sonosensitizer molecules 112 is linked to a carrier, such as a viral-like particle 118. Advantageously, the carrier 118 has intrinsic targeting capability; thus, there are no targeting agents linked to the carrier. In this non-limiting example, one or more IRDye® 700 112 molecules is conjugated to a virus-like particle.

With respect to FIG. 1C, one or more sonosensitizer molecules 133 is linked to a carrier 135. The carrier does not have an intrinsic targeting ability; thus, there may be a need to link one or more targeting agents 138 to the carrier. In this non-limiting example, one or more IRDye® 700 molecules 133 is conjugated to a carbon nanotube 135 and the nanotube is also ‘painted’ with one or more antibodies 138. The targeting agent 138 can be a small molecule.

i. Carrier Moiety

The present disclosure provides a sonosensitizer composition comprising IRDye® 700DX. The compositions comprising IRDye® 700DX may also comprise a carrier moiety. In certain aspects, the sonosensitizer compositions of the present disclosure comprise a nanocarrier, such as a virus-like particle, a nanoparticle, a nanotube (e.g., a carbon nanotube), a liposome, a quantum dot, or a combination thereof, that is attached to the sonosensitizing agent. The nanocarrier can be directed to the target cell or tissue of interest by passive targeting or directed targeting. With passive targeting the nanocarrier is transported to the target by convection (e.g., movement within fluids) or passive diffusion (e.g., movement across the cell membrane according to, for example, a concentration gradient or without the use of cellular energy) within the body. For directed targeting, a targeting agent can be attached to the surface of the nanocarrier for binding to its corresponding binding partner expressed at the target site.

a. Virus-like Particles

Provided herein are virus-like particles that can be used for delivering the sonosensitizer compositions to cells and tissues of the body. These particles can be produced from recombinant proteins that mimic specific viruses. The particles can be loaded with sonosensitizer compositions and agents (e.g., a plurality of IRDye® 700DX) and targeted to specific cells. For instance, the virus-like particles can deliver the agents to tumor cells, such as tumor cells from the lung, colon, ovary, kidney, skin, central nervous system, blood, prostate, breast and the like.

Viron-derived nanocarriers can be produced from papillomavirus capsids composed of L1 (major capsid protein) and L2 capsid (minor capsid protein) proteins. The viral-like particles of the present disclosure can be formed from about multiple assembled capsomers wherein each capsomer comprises L1 and L2 capsid proteins. The particles can have a stoichiometry of L1:L2 of about 15:1, about 10:1, or about 5:1. In other instances, the ratio is 15:1, 14:1, 13:1, 12:1, 11:1, 10:1, 9:1, 8:1, 7:1, 6:1, 5:1, 4:1, 3:1, 2:1, or 1:1.

In certain instance, there may be more than 1 IRDye® 700DX molecule per virus-like particle. In certain instances, there are 1, 5, 10, 15, 20, 25, 30, 35, 40, 45, 50, 55, 60, 65, 70, 75, 80, or more IRDye® 700DX molecules per virus-like particle.

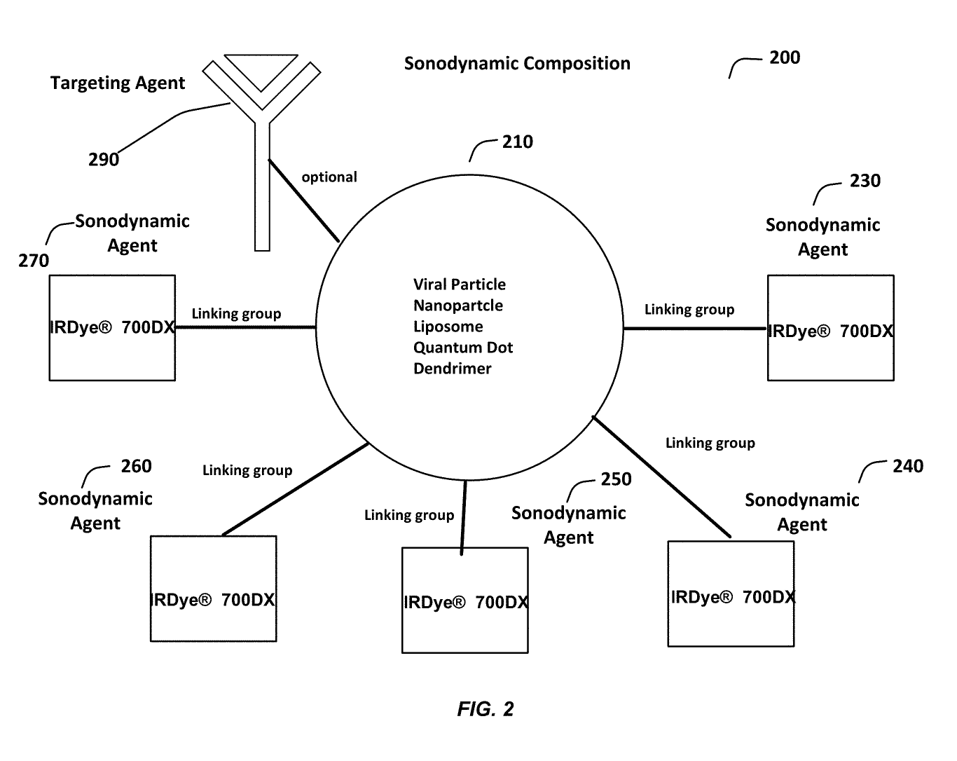

FIG. 2 shows an exemplary sonosensitizer composition 200 comprising IRDye® 700DX of the present disclosure. As shown therein, a viral particle 210 has covalently bound thereto IRDye® 700DX 230, 240, 250, 260, 160 and 270. An optional targeting agent 290 is attached (See Formula Ib below). The ratio is 1:5 viral particle to IRDye® 700DX. Of course, all ratios of viral particles to IRDye® 700DX molecules are included within the scope of this disclosure such as 1:5; 1:10; 1:50; 1:100; 1:500 and 1:1000 (and all ratios in-between) viral particle to IRDye® 700DX molecules.

In some embodiments, the papillomavirus is from a non-human vertebrate such as, but not limited to, an ungulate, canine, lapine, avian, rodent, simian, marsupial or marine mammal. In some embodiments, the papillomavirus is from a human. In other embodiments, the papillomavirus is selected from HPV-1, HPV-2, HPV-5, HPV-6, HPV-11, HPV-18, HPV-31, HPV-45, HPV-52, and HPV-58, bovine papillomavirus-1, bovine papillomavirus-2, bovine papillomavirus-4, cottontail rabbit papillomavirus, and rhesus macaque papillomavirus.

In some embodiments, the sonodynamic agent is encapsulated within the virus-like particle.

The virus-like particle can be generated by isolating and purifying capsid proteins produced in a host cell system, such as bacterial cell, yeast cell, insect cell or mammalian cell system. In some embodiments, the L1 and L2 proteins are intracellularly assembled. Alternatively, the particle can be generated by purifying the capsid proteins produced in an in vitro cell-free protein synthesis system. The capsid proteins can readily self-assemble into particles. For instance, L1 can spontaneously self-assemble into a 60 nm, 72-pentamer icosahedral structure that closely resembles a papillomavirus virion.

In some embodiments, viral capsid proteins L1 and/or L2 or fragments thereof (e.g., L1 peptides and/or L2 peptides) are coupled to IRDye® 700DX or to another carrier. In some instances, the capsid protein or peptide is coupled to IRDye® 700DX or the external surface of another carrier covalently or non-covalently. In some instances, the coupling comprises a covalent linker such as, but not limited to, an amide linker, a disulfide linker, a thioether linker, a hydrazone linker, a hydrazide linker, an imine or oxime linker, an urea or thiourea linker, an amidine linker, an amine linker, or a sulfonamide linker.

In some aspects, the capsid proteins are papilloma virus capsid proteins. For example, in some embodiments, the papilloma virus capsid proteins are non-human papilloma virus capsid proteins, such as bovine papilloma virus capsid proteins. In some aspects, the virus-like particles comprise human papilloma virus capsid proteins and do not cross-react with human papilloma virus (HPV) 16, HPV 18 or pre-existing antibodies specific for HPV. (See, WO 2015042325, incorporated herein by reference).

In some aspects, the virus-like particles comprise papilloma LI or L1/L2 proteins (e.g., of human, bovine, or other species). In some embodiments, the LI or L1/L2 VLPs do not cross-react with neutralizing antibodies to human papilloma virus (HPV) 16, HPV 18 or pre-existing antibodies specific for other HPVs. However, in some aspects, the virus-like particles comprise human papilloma virus capsid proteins of HPV 16.

In some aspects, the photosensitive molecules are conjugated to surface-exposed peptides of capsid proteins. In some aspects, the virus-like particles comprise LI capsid proteins or a combination of LI and L2 capsid proteins. In some aspects, the virus-like particles consist of LI capsid proteins.

In some aspects, the capsid proteins of a virus-like particle have modified immunogenicity and/or antigenicity. A non-limiting example of such a capsid protein is HPV16/31 LI capsid proteins (e.g., SEQ ID NO: 1). Virus-like particles that contain modified capsid proteins may be referred to herein as virus-like particles that contain modified immunogenicity and/or antigenicity compared to wild-type virus-like particles.

In some aspects, a virus-like particle comprises BPV LI capsid protein (e.g., SEQ ID NO: 2), a combination of BPV LI and BPV L2 capsid proteins. In some aspects, a virus-like particle comprises HPV LI capsid proteins, or a combination of HPV LI and HPV L2 capsid proteins. In some aspects, the HPV LI capsid protein is a variant HPV 16/31 LI protein having modified immunogenicity and/or antigenicity (e.g., SEQ ID NO: 1). Thus, in some aspects, a virus-like particle comprises or consists of variant HPV16/31 LI capsid proteins or a combination of variant HPV16/31 LI capsid proteins (e.g., SEQ ID NO: 1) and HPV L2 capsid proteins.

b. Nanoparticles or Nanotubes

Biodegradable or non-biodegradable polymers may be used to form nanoparticles or nanocarriers of the present disclosure. In certain embodiments, synthetic polymers are used, although natural polymers may be used and may have equivalent or even better properties, especially some of the natural biopolymers which degrade by hydrolysis, such as some of the polyhydroxyalkanoates. Examples of synthetic polymers include, but are not limited to, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, celluklose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(butyric acid), poly(valeric acid), and poly(lactide-co-caprolactone), cyclodextrins, and copolymers and blends thereof. As used herein, the term “derivatives” includes polymers having substitutions, additions of chemical groups and other modifications routinely made by those skilled in the art.

In particular embodiments, PLGA is used as the biodegradable polymer. Examples of biodegradable polymers useful in the present disclosure include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), and blends and copolymers thereof. Natural polymers include, but are not limited to, proteins such as albumin, collagen, gelatin and prolamines, for example, zein, and polysaccharides such as alginate, cellulose derivatives and polyhydroxyalkanoates, for example, polyhydroxybutyrate. The in vivo stability of the particles can be adjusted during the production by using polymers such as poly(lactide-co-glycolide) copolymerized with polyethylene glycol (PEG). Examples of non-biodegradable polymers include, but are not limited to, ethylene vinyl acetate, poly(meth)acrylic acid, polyamides, or copolymers or mixtures thereof.

In particular embodiments, the present disclosure provides nanocarriers or nanoparticles in the form of tubular bodies such as nanotubes. Nanotubes can be produced in a wide range of sizes and composed of a wide range of materials, or combination of materials. Nanotubes can be either hollow or solid and can be prepared having a highly monodisperse size distribution. In certain aspects, nanotubes of the present disclosure provide a means of delivering a payload, such IRDye® 700DX, to a required site. If necessary, nanotubes also provide a means of releasing the payload after delivery.

In certain aspects, one or more surfaces of the nanotube are functionalized to allow attachment of molecules to the surface. “Functionalized” nanotubes are nanotubes having at least one surface modified to allow for the directed delivery and/or controlled release of the nanotube payload. Nanotubes have distinct inner and outer surfaces, and their open geometry makes accessing and functionalizing these surfaces possible. Different chemical and/or biochemical functional groups can be applied to the inside and outside surfaces of the nanotube. Alternatively, one chemical/biochemical species can be applied to the inside surfaces of the nanotube, a second species to the outside surfaces and a third different species to the nanotube mouths. Methods used to functionalize a nanotube surface depend on the composition of the nanotube and are well known in the art. For example, functionalization of silica nanotubes is accomplished using silane chemistry. Here, different functional groups can be attached to the inside and outside surfaces of a nanotube by attaching a first group to the inner surface while the nanotubes are still embedded within the pores of the template.

c. Liposomes

In certain aspects, the carrier is a liposome. In one aspect, liposomes are artificial vesicles composed of concentric lipid bilayers separated by water-compartments and have been extensively investigated as drug delivery vehicles. Due to their structure, chemical composition and colloidal size, all of which can be well controlled by preparation methods, liposomes exhibit colloidal size, i.e., rather uniform particle size distributions in the range from 10 nm to 10 μm, and useful membrane and surface characteristics. Liposomes can deliver therapeutics and/or IRDye® 700DX to diseased tissues, for example, in circulation, and also rapidly enter the liver, spleen, kidneys and reticuloendothelial systems.

In some embodiments, the liposome comprises synthetic phospholipids such as, but not limited to, phosphatidyl cholines, e.g., dipalmitoylphosphatidy choline (DPPC), dimyristoyl phosphatidyl choline (DMPC), and distearoyl phosphatidyl choline (DSPC), and phosphatidyl glycerols, e.g., as dipalmitoyl glycerol (DPPG) or dimyristoyl phosphatidyl glycerol (DMPG). The liposome can also include a monosaccharide such as glucose or fructose. In some embodiments, the phospholipids are conjugated to a polyethylene glycol (PEG) molecule.

d. Quantum Dots

In certain aspect, the carrier is a quantum dot. Quantum dots are small molecular clusters having up to about a few hundred atoms. Quantum dots can have a size range of about 1 nm to about 20 nm in diameter. They are typically only a few nanometers in size. A quantum dot is typically composed of a semiconductor material or materials, metal(s), or metal oxides exhibiting a certain energy. A variety of materials may be utilized for construction of nanoparticles, including, but not limited to, TiO₂, Al₂O₃, AgBr, CdSe, CdS, CdSlSel, CuCl, CdTeₓSᵢ₋ₓ, ZnTe, ZnSe, ZnS, GaN, InGaN, InP, CdS/HgS/CdS, InAs/GaAs, Group II-VI, Groups III-V, and Groups I-VII semiconductors as well as Group IV metals and alloys. A quantum dot may also be surrounded by a material or materials having wider bandgap energies (for example, ZnS-capped CdS), and especially may be surrounded by those materials that improve biocompatibility of the nanoparticles.

There are a number of methods of making quantum dots. The synthesis of small semiconductor clusters in trioctylphosphine oxide (TOPO) at 300° C. has been shown to yield highly fluorescent (quantum yields >50%) small particles of a number of semiconductor materials, such as CdSe, InP and InAs. Growth conditions such as the length of time of crystallization, concentration of monomer, and temperature establish the size of the quantum dot and therefore the color of the light emitted from the quantum dot. (See, Green and O'Brien, Chem. Commun., 1999, 2235-41; and U.S. Pat. Nos. 5,909,670, 5,943,354, and 5,882,779). Quantum dots are commercially available from manufacturers such as Life Technologies, Nanoco Technologies, and Sigma-Aldrich.

In some embodiments, the nanoparticle comprises an inorganic material and/or a quantum dot. In some instances, the nanoparticle includes one or more materials selected from the group consisting of cadmium, zinc, magnesium, mercury, aluminum, gallium, indium, and thallium. Optionally, the nanoparticle can contain one or more materials selected from the group of cadmium, zinc, magnesium, mercury, aluminum, gallium, indium, or thallium.

In particular embodiments, the nanoparticle comprising an inorganic material can comprises a core and a shell, where the shell comprises a semiconductor overcoating the core. In certain embodiments the shell comprises a group II, III, IV, V, or VI semiconductor. In particular embodiments the shell comprises one or more materials selected from the group consisting of ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, and TlSb. In certain embodiments the nanoparticle comprises a CdSe core and a ZnS shell and a SiO2 hydrophilic coating.

e. Dendrimers

In some embodiments, the nanoparticle comprises a dendrimer. Dendrimers are typically nano-sized (1-100 nm) globular macromolecules with a unique architecture consisting of three distinct domains: a central core, a hyperbranched mantle and a corona with peripheral reactive functional groups. Dendrimers can be conveniently synthesized by convergent or divergent synthesis. The high level of control over the synthesis of dendritic architecture makes dendrimers a nearly perfect (spherical) nanocarrier with predictable properties. Numerous classes of dendrimers including polyamidoamine (PAMAM), polypropyleneimine (PPI), poly(glycerol-co-succinic acid), poly-L-lysine (PLL), melamine, triazine, poly(glycerol), poly[2,2-bis(hydroxymethyl)propionic acid] and poly(ethylene glycol) (PEG), as well as carbohydrate-based and citric-acid-based ones, have been developed for drug delivery. Among them, PAMAM- and PPI-based dendrimers have been some of the most widely investigated.

In certain aspects, the surface of the dendrimer is functionalized to allow attachment of molecules to the surface such as IRDye® 700DX and or a targeting agent.

f. Bicelle and or Micelle

In some embodiments, the nanoparticle comprises a bicelle. A bicelle is a self-assembled aggregate of phospholipid in water, that combines flat bilayer-like and curved micelle-like features.

In some embodiments, the nanoparticle comprises a micelle. A micelle is an aggregate (or supramolecular assembly) of surfactant molecules dispersed in a liquid colloid. A typical micelle in aqueous solution forms an aggregate with the hydrophilic “head” regions in contact with surrounding solvent, sequestering the hydrophobic single-tail regions in the micelle center.

ii. Targeting Agent

The present disclosure provides a sonosensitizer composition comprising IRDye® 700DX. In certain embodiments, the a sonosensitizer composition having IRDye® 700DX is attached to a nanocarrier and/or a targeting molecule. The targeting molecule can be entrapped within or associated with the surface of a nanocarrier (e.g., adsorbed or conjugated (directly or indirectly) to the nanocarrier surface), and/or otherwise associated with the nanocarrier to varying degrees (e.g., admixed with nanocarrier in a liquid suspension, admixed with the nanocarrier in a solid composition, for instance, co-lyophilized with the nanocarrier, etc.), among other possibilities. In some embodiments, at least two different targeting molecules are attached to a nanocarrier.

In some embodiments, the targeting molecule is an antibody, an antibody fragment, or an antibody mimetic that binds an antigen selected from the group consisting of, a gastrointestinal cancer cell surface antigen, a lung cancer cell surface antigen, a brain tumor cell surface antigen, a glioma cell surface antigen, a breast cancer cell surface antigen, an esophageal cancer cell surface antigen, a common epithelial cancer cell surface antigen, a common sarcoma cell surface antigen, an osteosarcoma cell surface antigen, a fibrosarcoma cell surface antigen, a melanoma cell surface antigen, a gastric cancer cell surface antigen, a pancreatic cancer cell surface antigen, a colorectal cancer cell surface antigen, a urinary bladder cancer cell surface antigen, a prostatic cancer cell surface antigen, a renal cancer cell surface antigen, an ovarian cancer cell surface antigen, a testicular cancer cell surface antigen, an endometrial cancer cell surface antigen, a cervical cancer cell surface antigen, a Hodgkin's disease cell surface antigen, a lymphoma cell surface antigen, a leukemic cell surface antigen and a trophoblastic tumor cell surface antigen.

In some embodiments, the targeting moiety is an antibody that binds an antigen selected from the group consisting of 5 alpha reductase, α-fetoprotein, AM-1, APC, APRIL, BAGE, β-catenin, Bc12, bcr-abl (b3a2), CA-125, CASP-8/FLICE, Cathepsins, CD19, CD20, CD21, CD23, CD22, CD38, CD33, CD35, CD44, CD45, CD46, CDS, CD52, CD55, CD59 (791Tgp72), CDC27, CDK4, CEA, c-myc, Cox-2, DCC, DcR3, E6/E7, EGFR, EMBP, Ena78, FGF8b and FGF8a, FLK-1/KDR, folic acid receptor, G250, GAGE-Family, gastrin 17, GD2/GD3/GM2, GnRH, GnTV, gp100/Pmel17, gp-100-in4, gp15, gp75/TRP-1, hCG, Heparanase, Her2/neu, HER3, Her4, HMTV, HLA-DR10, Hsp70, hTERT , IGFR1, IL-13R, iNOS, Ki 67, KIAA0205, K-ras, H-ras, N-ras, KSA, (CO17-1A), LDLR-FUT, MAGE Family (MAGE1, MAGE3, etc.), mammaglobin, MAP17, Melan-A/, MART-1, mesothelin, MIC A/B, MT-MMP's, such as MMP2, MMP3, MMPI, MMP9, Mox1, MUC-1, MUC-2, MUC-3, and MUC-4, MUM-1, NY-ESO-1, Osteonectin, p15, P170/MDR1, p53, p97/melanotransferrin, PAI-1, PDGF, plasminogen (uPA), PRAME, probasin, progenipoietin, PSA, PSM, RAGE-1, Rb, RCAS1, SART-1, SSX gene, family, STAT3, STn, TAG-72, TGF-α, TGF-β, and thymosin β, 15, nucleolin, Cal5-3, astro Intestinal Tumor Antigen (Ca19-9), ovarian tumor antigen (Ca125), tag72-4 antigen (CA72-4) and carcinoembryonic antigen (CEA).

In some embodiments, the targeting molecule specifically binds to an antigen such as a tumor antigen, bacterial antigen viral antigen, and fungal antigen. The targeting molecule can recognize tumor antigens such as, but not limited to: (a) cancer-testis antigens such as NY-ESO-1, SSX2. SCP1 as well as RAGE, BAGE, GAGE and MAGE family polypeptides, for example, GAGE-1, GAGE-2, MAGE-1. MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g. melanoma, pancreatic cancer and colorectal cancer), CDK4 (associated with, e.g. melanoma), MUM1 (associated with, e.g., melanoma), caspase-8 (associated with, e.g., head and neck cancer), CIA 0205 (associated with, e.g., bladder cancer), HLA-A2-R 1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT. (c) over-expressed antigens, for example, Galectin 4 (associated with, e.g., colorectal cancer), galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g. renal cancer), aldolase A (associated with, e.g., lung cancer), PRAME (associated with, e.g. melanoma). HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g. prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), which also may be coupled to carrier proteins (e.g., KLH).

Other tumor antigens include p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, (CT7, 43-9F, 5T4, 791 Tgp72, beta-HCG, BCA225, BTAA, CA 125, CA 15-3 (CA 27.29\BCAA). CA 195, CA 242, CA-50, CAM43, CD68\KP1, CO-029, FGF-5, Ga733 (EpCAM), 1HTgp-175, M344, MA-50, MG7-Ag, MOV18, NB/70K, NY-CO-1. RCAS1, SDCCAG16, TA-90 (Mac-2 binding protein\cyclophilin C-associated protein), TAAL6, TAG72, TLP, TPS, and the like.

In certain aspects, other targeting moieties include an antigen, a ligand, a protein, a peptide, a carbohydrate, a nucleic acid, or a small molecule. In one aspect, these targeting agents specifically bind to the predetermined target cell. The targeting moiety can be less than the molecular weight of an antibody. In some aspects, the probe is less than about 50 kDa, e.g., about 49 kDa, 45 kDa, 4-kDa, 35 kDa, 30 kDa, 25 kDa, 20 kDa, 15 kDa, 10 kDa, 5 kDa, 1 kDa, or less than 1 kDa. The probe can be less than about 10 kDa, e.g., 9 kDa, 8 kDa, 7 kDa, 6 kDa, 5 kDa, 4 kDa, 3 kDa, 2 kDa, 1 kDa, or less than 1 kDa.

In some embodiments, the targeting molecule is a small molecule such as a carbohydrate. Carbohydrates may be natural or synthetic. A carbohydrate may be a derivatized natural carbohydrate. In some embodiments, the carbohydrate comprises monosaccharide or disaccharide, including but not limited to, glucose, fructose, galactose, ribose, lactose, sucrose, maltose, trehalose, cellbiose, mannose, xylose, arabinose, glucoronic acid, galactoronic acid, mannuronic acid, glucosamine, galatosamine, or neuramic acid. In some embodiments, the carbohydrate is a polysaccharide, such as, but not limited to, pullulan, cellulose, microcrystalline cellulose, hydroxypropyl methylcellulose (HPMC), hydroxycellulose (HC), methylcellulose (MC), dextran, cyclodextran, glycogen, starch, hydroxyethyl starch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate is a sugar alcohol, such as, but not limited to mannitol, sorbitol, xylitol, erythritol, maltitol, or lactitol.

In certain aspects, the present disclosure provides a sonosensitizer composition comprising IRDye® 700DX together with a small molecule. The compositions comprising IRDye® 700DX may also comprise a carrier moiety with a small molecule appended to the carrier moiety. In certain aspects, the sonosensitizer compositions of the present disclosure comprise a nanocarrier, such as a virus-like particle, a nanoparticle, a nanotube (e.g., a carbon nanotube), a liposome, a quantum dot, or a combination thereof, that is attached to the sonosensitizing agent and a small molecule.

In some instances, the small molecule is selected from the group of a VEGFR inhibitor, a TNFR1 inhibitor, a growth factor receptor inhibitor and combinations thereof. In some instances, the small molecule VEGFR inhibitor is selected from the group of pazopanib, semaxanib, axitinib, cabozantinib, aflibercept, brivanib, tivozanib, ramucirumab, motesanib, vatalanib, cediranib, and combinations thereof. Alternatively, the probe can be a member selected from the group of DTPA-octreotide, [Gluc-Lys]-TOCA, galacto-RGD, AH111585, RGD-K5, FPPRGD2, RP-527, BZH3, [DTPA-Lys40]-Exendin-4, and Tc-NT-X1.

In some instances, the small molecule VEGFR inhibitor is selected from the group of pazopanib, semaxanib, axitinib, cabozantinib, aflibercept, brivanib, tivozanib, ramucirumab, motesanib, vatalanib, cediranib, and combinations thereof.

In certain aspects, the targeting moiety is a small molecular weight protein, ligand, peptide, cyclic peptide, small molecule, and analogs thereof that bind to the cell surface of the target cell. In some aspects, the targeting moeity is EGF, YC-27, cRGDfK, vasoactive intestinal peptide, gastrin-releasing peptide, AH111585, FPPRGD2, PK11195, SPARC, bombesin, neurotensin, substance P, somatostatin, cholecystokinin, glucagon-like peptide-1, neuropeptide Y, octreotide, DOTA-TOC, DOTA-TATE, exendin-4, soricidin, SOR-13, SOR-C27, a small molecule VEGFR inhibitor, e.g., pazopanib, semaxanib, axitinib, cabozantinib, aflibercept, brivanib, tivozanib, ramucirumab, motesanib, vatalanib, cediranib, and combinations thereof, a small molecule TNF1R inhibitor, a growth factor receptor inhibitor, DTPA-octreotide, [Gluc-Lys]-TOCA, galacto-RGD, AH111585, RGD-K5, FPPRGD2, RP-527, BZH3, [DTPA-Lys⁴⁰]-Exendin-4, Tc-NT-X1, analogs thereof or derivatives thereof.

YC-27 is a prostate specific membrane antigen-specific (PSMA-specific) small molecule. PSMA is also known as folate hydrolase I or glutamate carboxypeptidease II. See, e.g., U.S. Patent Application Publication No. 2012/0009121, Chen et al., Biochem Biophys Res Commun, 390(3):624-629, 2009 and Kovar et al., Prostate Cancer, 2014, article ID 104248, 10 pages.

The small molecule PK-11195 is an isoquinoline carboxamide that selectively binds to the peripheral benzodiazepine receptor. PK-11195 can act as a GABA-A antagonist. One skilled in the art will recognize that PK-11195 is also known as 1-(2-chlororphenyl)-N-methyl-N-(1-methylpropyl)-1-isoquinoline carboxamide.

Edotreotide, DOTA⁰-Phe¹-Tyr³ or DOTA-TOC is a small molecule that can selectively bind to somatostatin receptors.

Non-limiting examples of small molecule VEGF receptor inhibitors include pazopanib (GW786034B; GlaxoSmithKline), GW654652 (GlaxoSmithKline), semaxanib (SU5416; Sugen), axitinib (INLYTA®, Pfizer), cabozantinib (COMTRIQ™, XL184; Exelixis), aflibercept (Sanofi-Aventis); brivanib (BMS-582664; Bristol-Myers Squibb), tivozanib (AV-651; AVEO Pharmaceuticals), ramucirumab (CYRAMZA™; Eli Lilly and Company), motesanib (Takeda Pharmaceutical Company Limited), vatalanib (PTK787/ZK222584; Bayer Schering and Novartis), and cediranib (RECENTIN™, AZD 2171; AstraZeneca).

In some aspects, IRDye® 700DX is conjugated to a fluorophore. Suitable fluorophores include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.TM.Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Oregon Green; Cy 3; Cy 5; Cy 5.5; Cy 7; IRDye® 700; IRDye® 800CW; La Jolla Blue; phthalo cyanine; naphthalo cyanine ATT0647; and IRDye® 680LT.

B. Conjugating IRDye® 700DX to a Carrier

The present disclosure provides sonosensitizer compositions comprising IRDye® 700DX. The compositions comprising IRDye® 700DX may also comprise (optionally comprises) a carrier moiety. IRDye° 700DX (LI-COR, Lincoln, Nebr.) can be covalently conjugated to nanocarrier. In some aspects, the carriers are conjugated to IRDye® 700DX according to the manufacturer's protocols and kits. Detailed descriptions of methods for producing IRDye® 700DX-conjugates are found in, e.g., Kovar et al, Biochemistry—Faculty Publications, 2007, paper 9; Mitsunaga et al., Nature Medicine, 2011, 17:1685-1691; Peng et al., Proceedings of SPIE, 2006, 6097; U.S. Patent Nos. 7,005,518 and 8,524,239; and U.S. Patent Application Publication No. 2013/0336995, the disclosures of each are herein incorporated in their entirety for all purposes.

Methods of linking dyes to various types of nanocarrier and/or targeting agents are well-known in the art. For a thorough review of, e.g., oligonucleotide labeling procedures, see R. Haugland in Excited States of Biopolymers, Steiner ed., Plenum Press (1983), Fluorogenic Probe Design and Synthesis: A Technical Guide, PE Applied Biosystems (1996), and G. T. Herman, Bioconjugate Techniques, Academic Press (1996).

IRDye® 700DX having a linker is shown below in Formula I:

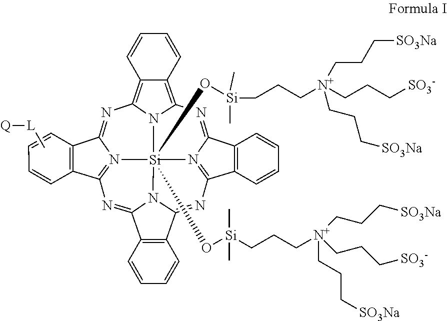

In certain aspects, Q comprises a reactive group for attachment to a a nanocarrier and/or targeting agent. Preferably, Q comprises a reactive group that is reactive with a carboxyl group, an amine, or a thiol group on the a nanocarrier and/or targeting agent.

Suitable reactive groups include, but are not limited to, an activated ester, an acyl halide, an alkyl halide, an optionally substituted amine, an anhydride, a carboxylic acid, a carbodiimide, a hydroxyl, iodoacetamide, an isocyanate, an isothiocyanate, a maleimide, an NHS ester, a phosphoramidite, a sulfonate ester, a thiol, or a thiocyanate (See Table 1 below).

L, in Formula I, is selected from a direct link, or a covalent linkage, wherein the covalent linkage is linear or branched, cyclic or heterocyclic, saturated or unsaturated, having 1 60 atoms selected from C, N, P, O, wherein L can have additional hydrogen atoms to fill valences (in addition to the 1-60 atoms), wherein the linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds. In certain instances, L comprises a terminal amino, carboxylic acid, or sulfhydryl group and is represented as -L-NH₂, or -L-C(O)OH or -L-SH.

The linker “L-Q” can include a phosphoramidite group, an NHS ester, an activated carboxylic acid, a thiocyanate, an isothiocyanate, a maleimide and an iodoacetamide.

In certain aspects, the linker L comprises a -(CH₂)n- group, wherein r is an integer from 1 to 10, preferably n is an integer from 1 to 5, such as 1 to 4, or 1, 2, 3, 4, or 5, and L-Q comprises a —O—(CH₂)n-NH₂, or O—(CH₂)n-C(O)OH or O—(CH₂)ₙ-SH.

In one aspect, L-Q in Formula I, is —O—(CH₂)₃—OC(O)—NH—(CH₂)₅—C(O)O—N— succinimidyl as shown below:

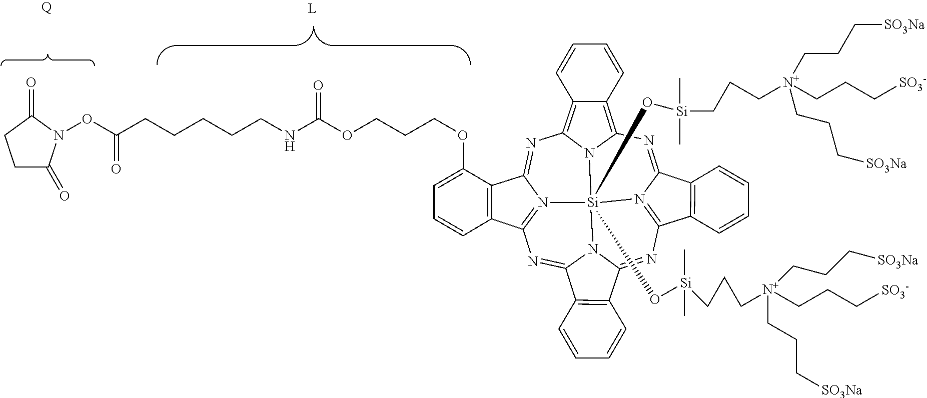

In certain instances, the dye is reacted with a nanocarrier and/or targeting agent having a primary amine to make a stable amide bond. In other aspects, a maleimide and a thiol can react together and make a thioether. Alkyl halides react with amines and thiols to make alkylamines and thioethers, respectively. Any derivative providing a reactive moiety that can be conjugated to a a nanocarrier and/or targeting agent can be utilized herein. As is known in the art, moieties comprising a free amino group, a free carboxylic acid group, or a free sulfhydryl group provide useful reactive groups for protein conjugation. For example, a free amino group can be conjugated to proteins via glutaraldehyde cross-linking, or via carbodiimide cross-linking to available carboxy moieties on the protein. Also, a linker with a free sulfhydryl group can be conjugated to proteins via maleimide activation of the protein, e.g., using sulfosuccinimidyl-4-(N-maleimidomethyl)cyclohexane-1-carboxylate (Sulfo-SMCC), then linkage to the sulfhydryl group.

When linking a dye having a carboxylic acid group for attachment to an amine containing a nanocarrier and/or targeting agent molecule, the carboxylic acid can first be converted to a more reactive form using an activating reagent, to form for example, a N-hydroxy succinimide (NHS) ester or a mixed anhydride. The amine-containing a nanocarrier and/or targeting agent is treated with the resulting activated acid to form an amide linkage. One of skill in the art will recognize that alternatively, the NHS ester can be on the a nanocarrier and/or targeting agent and the amine can be on the dye.

In other aspects, the linker is a member selected from the group of a PEG, a block copolymer of PEG-polyurethane and a PEG-polypropylene. In yet other aspects, the linker is a member selected from the group of a polysaccharide, a polypeptide, an oligosaccharide, a polymer, a co-polymer and an oligonucleotide.

The linker L can have the formula:

C¹—Y¹—X²—

wherein: X¹ is a member selected from the group of a bivalent radical, a direct link, oxygen, an optionally substituted nitrogen and sulfur; Y¹ is a member selected from the group of a direct link and C₁-C₁₀ alkylene optionally interrupted by a heteroatom; and X² is a member selected from the group of a bivalent radical, a direct link, oxygen, an optionally substituted nitrogen and sulfur.

Preferably, the bivalent radical of X¹ and X² are each independently selected from the group of a direct link, optionally substituted alkylene, optionally substituted alkyleneoxycarbonyl, optionally substituted alkylenecarbamoyl, optionally substituted alkylenesulfonyl, optionally substituted alkylenesulfonylcarbamoyl, optionally substituted arylene, optionally substituted arylenesulfonyl, optionally substituted aryleneoxycarbonyl, optionally substituted arylenecarbamoyl, optionally substituted arylenesulfonylcarbamoyl, optionally substituted carboxyalkyl, optionally substituted carbamoyl, optionally substituted carbonyl, optionally substituted heteroarylene, optionally substituted heteroaryleneoxycarbonyl, optionally substituted heteroarylenecarbamoyl, optionally substituted heteroarylenesulfonylcarbamoyl, optionally substituted sulfonylcarbamoyl, optionally substituted thiocarbonyl, a optionally substituted sulfonyl, and optionally substituted sulfinyl.

Alternatively, the linker is —(CH₂)ᵣ-, wherein r is an integer from 1 to 50.

The reactive Q group of Formula I reacts with a complementary group on the nanocarrier to form a compound of Formula Ia:

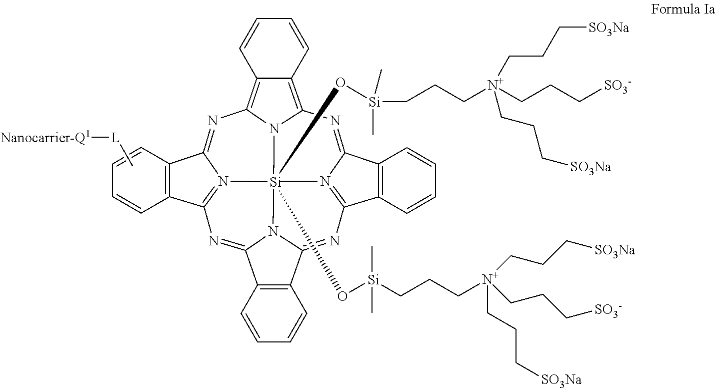

In Formula Ia, the reactive Q group of Formula I reacts with a complementary group on the nannocarrier and forms a covalent linkage Q¹. The a nanocarrier and/or targeting agent is then attached covalently to the linker.

In one aspect, IRDye® 700DX has an NHS ester as above and the nanocarrier has an amine, which reacts to form an amide:

IRDye® 700DX-O—(CH₂)₃—OC(O)—NH—(CH2)5—C(O)NH-Nanocarrier

Selected example of reactive functionalities useful for the attaching the dye to the a nanocarrier and/or targeting agent are shown in Table I, wherein the bond results from the reaction of the dye (e.g., detecting agent or sonosensitizing agent) with the a nanocarrier and/or targeting agent. Those of skill in the art will know of other bonds suitable for use in the present disclosure.

TABLE 1

| A | B | |
|---|---|---|
| Reactive | Complementary | |
| functionality Q on the | group on the | C |
| phthalocyanine dye | Nanocarrier | The resulting bond Q¹ |
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | Hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| anhydrides | alcohols/phenols | esters |
| anhydrides | amines/anilines | carboxamides/imides |
| aryl halides | Thiols | thiophenols |
| aryl halides | Amines | aryl amines |
| aziridines | thiols | thioethers |
| boronates | glycols | boronate esters |
| activated carboxylic acids | amines/anilines | carboxamides |
| activated carboxylic acids | alcohols | esters |
| activated carboxylic acids | hydrazines | hydrazides |
| carbodiimides | carboxylic acids | N-acylureas or |
| | | anhydrides |
| diazoalkanes | carboxylic acids | esters |
| epoxides | thiols (amines) | thioethers (alkyl amines) |
| epoxides | carboxylic acids | esters |
| haloacetamides | Thiols | thioethers |
| haloplatinate | amino | platinum complex |
| haloplatinate | heterocycle | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | Alcohols | phosphite esters |
| silyl halides | alcohols | silyl ethers |
| sulfonate esters | amines/anilines | alkyl amines |
| sulfonyl halides | amines/anilines | sulfonamides |

*Activated esters, as understood in the art, generally have the formula —COM, where M is a good leaving group (e.g. succinimidyloxy (—OC₄H₄O₂) sulfosuccinimidyloxy (—OC₄H₃O₂SO₃H), -1-oxybenzotriazolyl (—OC₆H₄N₃); 4-sulfo-2,3,5,6-tetrafluorophenyl; or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates.

In some aspects, the covalent linkage Q¹ between the linker and nanocarrier (Column “C”) is selected from the group of a direct bond, an amide bond, an ester bond, an ether bond, an oxime bond, a phosphate ester bond, a sulfonamide bond, a thioether bond, a thiourea bond, and an urea bond. In an alternative embodiment, the “A” reactive functional group is on the a nanocarrier and/or targeting agent and the complementary functional group “B” in on the dye.

In other aspects, IRDye® 700DX dye is linked to nanocarrier by click chemistry. Click chemistry uses simple, robust reactions, such as the copper-catalyzed cycloaddition of azides and alkynes, to create intermolecular linkages. For a review of click chemistry, see, e.g., Kolb, H. C.; Finn, M. G.; Sharpless, K. B. Angew. Chem. 2001, 40, 2004.

Connection (or ligation) of two fragments to make a larger molecule or structure is often achieved with the help of so-called click chemistry described by Sharpless et al., Angew. Chem., Int. Ed. 40: 2004 (2001). This term is used to describe a set of bimolecular reactions between two different reactants such as azides and acetylenes. The formation of 1,2,3-triazoles in 1,3-dipolar cycloaddition of azides to a triple bond is known, but because the activation energy of acetylene-azide cycloaddition is relatively high, the reaction is slow under ambient conditions.

The utility of the reaction of azides with alkynes was expanded by the discovery of Cu (I) catalysis. 1,3-cycloaddition of azides to terminal acetylenes in the presence of catalytic amounts of cuprous salts is facile at room temperature in organic or aqueous solutions.

U.S. Pat. No. 7,807,619 to Bertozzi et al. teaches modified cycloalkyne compounds and method of use of such compounds in modifying biomolecules. Bertozzi et al. teach a cycloaddition reaction that can be carried out under physiological conditions. As disclosed therein, a modified cycloalkyne is reacted with an azide moiety on a target biomolecule, generating a covalently modified biomolecule.

A skilled artisan will appreciate that the description above primarily describes conjugating IRDye® 700DX to a nanocarrier. However, the description is equally applicable to conjugating IRDye® 700DX to a targeting agent or alternatively, a nanocarrier to a targeting agent such as an antibody or small molecule as shown in Formula Ib below:

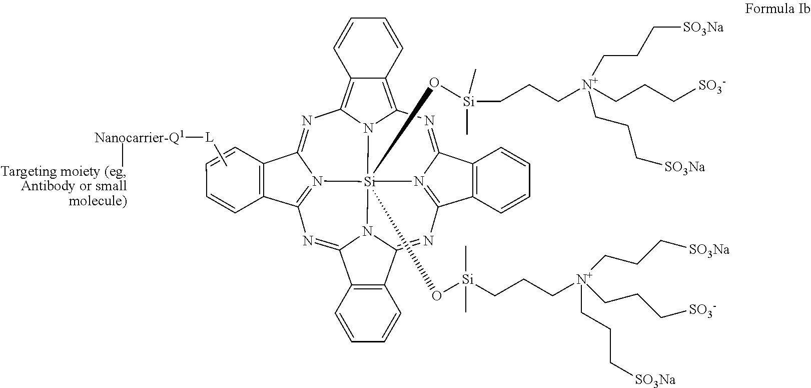

C. Combination Treatment

In certain instances, the sonosensitizer compositions of the disclosure are administered in combination with a known chemotherapeutic agent. For example, in certain aspects, the sonosensitizer compositions of the disclosure are administered in combination with a chemoprotective agent. Chemoprotective agents act to protect the body or minimize the side effects of chemotherapy. Non-limiting agents include radioactive isotopes; chemotherapeutic agents or drugs (e.g., methotrexate, Adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof. Other examples of such agents include, but are not limited to, amfostine, mesna, and dexrazoxane.

In some aspects of the disclosure, the sonosensitizer compositions are administered in combination with radiation therapy. Radiation is commonly delivered internally (implantation of radioactive material near cancer site) or externally from a machine that employs x-ray or gamma-ray radiation. Where the combination therapy further comprises radiation treatment, the radiation treatment may be conducted at any suitable time so long as a beneficial effect from the co-action of the combination of the sonosensitizer compositions and/or therapeutic agents and radiation treatment is achieved. For example, in appropriate cases, the beneficial effect is still achieved when the radiation treatment is temporally removed from the administration of the therapeutic agents, perhaps by days or even weeks.

D. Methods of Treatment and Administration

In certain instances, the sonosensitizer compositions of the disclosure are also useful for sonodestruction of normal or malignant animal cells, as desired. Thus, the disclosure further provides the use of the sonosensitizer compositions of the disclosure for in vivo, ex-vivo or in vitro killing of cells or infectious agents such as bacteria, viruses, parasites and fungi in a biological product, e.g. blood. Use of the sonosensitizer compositions accordingly comprises treating the infected sample with the compositions followed by ultrasound irradiation.

In certain instances, the sonosensitizer compositions of the disclosure treat solid tumors. Solid tumor included cancers such as lung, breast, bladder, ovarian, pancreatic, skin, esophagus, stomach, liver, colon and prostate cancer.

In certain instances, the sonosensitizer compositions of the disclosure can be used for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue. In certain instances, the sonosensitizer compositions of the disclosure can be used for the treatment of polyps.

In yet other aspects, the present disclosure provides methods and compositions for sonodynamic therapy (SDT) of target cells, tissues, and organs in a subject. In some instances, the target cells are cells of a solid tumor. In other instances, the target cells are located in the vasculature of the subject. SDT is a two-step treatment process that can be used in a wide variety of cancers and diseased tissue and organs. The first step in this therapy is carried out by administering a sonosensitizing agent systemically by ingestion or injection, or topically applying the compound to a specific treatment site on a subject, followed by the second step of illuminating the treatment site with energy having a wavelength or waveband corresponding to a characteristic absorption waveband of the sonosensitizing agent. The ultrasound activates the sonosensitizing agent, causing singlet oxygen radicals and other reactive oxygen species (superoxide) to be generated, leading to a number of biological effects that destroy the abnormal or diseased tissue, which has absorbed the sonosensitizing agent. The depth and volume of the cytotoxic effect (e.g., apoptotic effect or necrosis) on the abnormal tissue, such as a cancerous tumor or leaking blood vessel, depends in part on the depth of the ultrasound penetration into the tissue, the sonosensitizing agent concentration and its cellular distribution, and the availability of molecular oxygen, which will depend upon the vasculature system supplying the tumor, tissue, or organ.

In yet other aspects, the death of the diseased cell stimulates the immune system to create an immunogenic response to the sonodynamic therapy.

In yet other aspects, the depth of the tumor or tissue to be irradiated from the ultrasound transducer is greater than about 1 cm. In certain instances, the tissue is about 1 cm to 100 cm or about 5 cm to about 50 cm or about 1 cm to about 30 cm from the transducer such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 cm. In a typical application, the ultrasound transducer is in contact with the skin directly above the issue to be irradiated with the ultrasound.

In certain instances, the present disclosure provides methods for treatment, wherein for example, a tumor is treated using the therapeutic agent and thereafter, imaged to ascertain the extent of treatment. The treatment can be repeated until the tumor is destroyed or the site of treatment is satisfactorily complete. In certain instances, the methods include, injecting the composition, treating the tumor using sonodynamic therapy and thereafter imaging to ascertain the extent of treatment.

The sonosensitizer compositions of this disclosure can be administered sublingually, orally, parenterally, by inhalation spray, topically, rectally, nasally, buccally, vaginally or via an implanted reservoir. In certain instances, the administration is by oral administration or by injection, as deemed appropriate by those of skill in the art for bringing the compositions of the disclosure into optimal contact with the target tissue.

In certain aspects, the administration of the sonosensitizer compositions comprise systemic administration to the subject, local administration to a tumor, or administration to a surgical site.

The method of the present disclosure provides for administering to the subject a therapeutically effective amount of a targeted sonosensitizing agent. The composition can be administered systemically by ingestion or injection, or locally administered to a target tissue site or to a surgical site. The agent can bind to one or more types of target cells or tissues, such as circulating tumor cells or cells of a solid tumor. When exposed to ultrasonic waves, the agent absorbs the energy, causing substances to be produced that impair or destroy the target cells or tissues via heat, vibration or apoptosis. Preferably, the compound is nontoxic to the subject to which it is administered or is capable of being formulated in a nontoxic composition that can be administered to the subject. In addition, following exposure to ultrasound, the compound in any resulting degraded form is also preferably nontoxic.

E. Ultrasound

In one aspect, the method of the present disclosure provides an ultrasonic wave to the sonosensitizer composition once administered. The method comprises applying the ultrasonic wave to a diseased cell that generates acoustic cavitation and may induce light emission. Without being bound by any particular theory, if light emission does occur, then there is a ‘transduction’ from an ultrasonic energy source to an optical energy source which then interacts with IRDye® 700. Alternatively, there may be a ‘mechanic’ absorbance by IRDye® 700 (e.g., rotational or vibrational energy states) that ‘donates’ this absorbed energy to an ‘acceptor’ electronic singlet state of IRDye® 700, which then relaxes to an electronic triplet state of IRDye® 700. However, the actual mechanism of SDT is not really known.

In one aspect, the ultrasonic wave can be applied at a frequency greater than 20 kHz, such as about 0.1 MHz (100 kHz) to about 30 MHz, such as about 1.0 MHz to about 5.0 MHz or about 1.0 MHz to about 2.0 MHz. For example, the ultrasonic wave can be applied at 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 MHz.

In one aspect, the method of the present disclosure provides an ultrasonic wave applied at a power density of about 0.01 W/cm² to about 12 W/cm², such as a power density of about 1.0 W/cm² to about 6 W/cm² or about 1.2 W/cm² to about 3.8 W/cm². For example, the ultrasonic wave can be applied at 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 77, 8, 9, 10, 11, 12 W/cm².

In certain instances, the duration of the pulsing treatment can be in seconds, minutes, hours or even days. The treatment can be a continuous ultrasound wave, or a pulsing treatment with the pulse width from microseconds, milliseconds to seconds. If it is a pulsing treatment, it can be pulses of a square wave, a triangle wave, a rectangular wave, a sine wave, a saw tooth wave, or other different waveform shapes and geometries. The duty cycle (the percentage of time when the ultrasound energy is “on”) can be from 1% to 99% of the duration period. Duty cycle is the fraction of one period in which a signal or system is active. Duty cycle is commonly expressed as a percentage (1% to 99%). A period is the time it takes for a signal to complete an on-and-off cycle. As a formula, a duty cycle (%) may be expressed as: D=PW/T×100%, wherein D is the duty cycle, PW is pulse width and T is total time. Thus, a 20% duty cycle means the signal is on 20% of the time, but off 80% of the time.

As a skilled person will appreciate, the duration of treatment, duty cycle, pulsing waveform, and peak pulse power combine to generate the overall sonodynamic energy dosage to the agent at the treated area, leading to the sonodynamic treatment effect (SDT).

In certain instances, the ultrasonic wave is applied for milliseconds to minutes, hours and days (“the duration of treatment”). In certain aspects, the amount or duration of the pulse is about 1 second to 100 seconds; or about 1 to about 200 seconds; or about 1 to about 300 seconds; or about 1 to about 400 seconds; or about 1 to about 500 seconds; or about 1 to about 600 seconds. For example, the ultrasonic wave can be applied for approximately 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 sec, 61 sec, 62 sec, 63 sec, 64 sec, 65 sec, 66 sec, 67 sec, 68 sec, 69 sec, 70 sec, 71 sec, 72 sec, 73 sec, 74 sec, 75 sec, 76 sec, 77 sec, 78 sec, 79 sec, 80 sec, 81 sec, 82 sec, 83 sec, 84 sec, 85 sec, 86 sec, 87 sec, 88 sec, 89 sec, 90 sec, 91 sec, 92 sec, 93 sec, 94 sec, 95 sec, 96 sec, 97 sec, 98 sec, 99 sec, 100 sec, 101 sec, 102 sec, 103 sec, 104 sec, 105 sec, 106 sec, 107 sec, 108 sec, 109 sec, 110 sec, 111 sec, 112 sec, 113 sec, 114 sec, 115 sec, 116 sec, 117 sec, 118 sec, 119 sec, 120 sec, 121 sec, 122 sec, 123 sec, 124 sec, 125 sec, 126 sec, 127 sec, 128 sec, 129 sec, 130 sec, 131 sec, 132 sec, 133 sec, 134 sec, 135 sec, 136 sec, 137 sec, 138 sec, 139 sec, 140 sec, 141 sec, 142 sec, 143 sec, 144 sec, 145 sec, 146 sec, 147 sec, 148 sec, 149 sec, 150 sec, 151 sec, 152 sec, 153 sec, 154 sec, 155 sec, 156 sec, 157 sec, 158 sec, 159 sec, 160 sec, 161 sec, 162 sec, 163 sec, 164 sec, 165 sec, 166 sec, 167 sec, 168 sec, 169 sec, 170 sec, 171 sec, 172 sec, 173 sec, 174 sec, 175 sec, 176 sec, 177 sec, 178 sec, 179 sec, 180 sec, 181 sec, 182 sec, 183 sec, 184 sec, 185 sec, 186 sec, 187 sec, 188 sec, 189 sec, 190 sec, 191 sec, 192 sec, 193 sec, 194 sec, 195 sec, 196 sec, 197 sec, 198 sec, 199 sec, 200 sec, 201 sec, 202 sec, 203 sec, 204 sec, 205 sec, 206 sec, 207 sec, 208 sec, 209 sec, 210 sec, 211 sec, 212 sec, 213 sec, 214 sec, 215 sec, 216 sec, 217 sec, 218 sec, 219 sec, 220 sec, 221 sec, 222 sec, 223 sec, 224 sec, 225 sec, 226 sec, 227 sec, 228 sec, 229 sec, 230 sec, 231 sec, 232 sec, 233 sec, 234 sec, 235 sec, 236 sec, 237 sec, 238 sec, 239 sec, 240 sec, 241 sec, 242 sec, 243 sec, 244 sec, 245 sec, 246 sec, 247 sec, 248 sec, 249 sec, 250 sec, 251 sec, 252 sec, 253 sec, 254 sec, 255 sec, 256 sec, 257 sec, 258 sec, 259 sec, 260 sec, 261 sec, 262 sec, 263 sec, 264 sec, 265 sec, 266 sec, 267 sec, 268 sec, 269 sec, 270 sec, 271 sec, 272 sec, 273 sec, 274 sec, 275 sec, 276 sec, 277 sec, 278 sec, 279 sec, 280 sec, 281 sec, 282 sec, 283 sec, 284 sec, 285 sec, 286 sec, 287 sec, 288 sec, 289 sec, 290 sec, 291 sec, 292 sec, 293 sec, 294 sec, 295 sec, 296 sec, 297 sec, 298 sec, 299 sec, 300 sec, 301 sec, 302 sec, 303 sec, 304 sec, 305 sec, 306 sec, 307 sec, 308 sec, 309 sec, 310 sec, 311 sec, 312 sec, 313 sec, 314 sec, 315 sec, 316 sec, 317 sec, 318 sec, 319 sec, 320 sec, 321 sec, 322 sec, 323 sec, 324 sec, 325 sec, 326 sec, 327 sec, 328 sec, 329 sec, 330 sec, 331 sec, 332 sec, 333 sec, 334 sec, 335 sec, 336 sec, 337 sec, 338 sec, 339 sec, 340 sec, 341 sec, 342 sec, 343 sec, 344 sec, 345 sec, 346 sec, 347 sec, 348 sec, 349 sec, 350 sec, 351 sec, 352 sec, 353 sec, 354 sec, 355 sec, 356 sec, 357 sec, 358 sec, 359 sec, 360 sec, 361 sec, 362 sec, 363 sec, 364 sec, 365 sec, 366 sec, 367 sec, 368 sec, 369 sec, 370 sec, 371 sec, 372 sec, 373 sec, 374 sec, 375 sec, 376 sec, 377 sec, 378 sec, 379 sec, 380 sec, 381 sec, 382 sec, 383 sec, 384 sec, 385 sec, 386 sec, 387 sec, 388 sec, 389 sec, 390 sec, 391 sec, 392 sec, 393 sec, 394 sec, 395 sec, 396 sec, 397 sec, 398 sec, 399 sec, 400 sec, 401 sec, 402 sec, 403 sec, 404 sec, 405 sec, 406 sec, 407 sec, 408 sec, 409 sec, 410 sec, 411 sec, 412 sec, 413 sec, 414 sec, 415 sec, 416 sec, 417 sec, 418 sec, 419 sec, 420 sec, 421 sec, 422 sec, 423 sec, 424 sec, 425 sec, 426 sec, 427 sec, 428 sec, 429 sec, 430 sec, 431 sec, 432 sec, 433 sec, 434 sec, 435 sec, 436 sec, 437 sec, 438 sec, 439 sec, 440 sec, 441 sec, 442 sec, 443 sec, 444 sec, 445 sec, 446 sec, 447 sec, 448 sec, 449 sec, 450 sec, 451 sec, 452 sec, 453 sec, 454 sec, 455 sec, 456 sec, 457 sec, 458 sec, 459 sec, 460 sec, 461 sec, 462 sec, 463 sec, 464 sec, 465 sec, 466 sec, 467 sec, 468 sec, 469 sec, 470 sec, 471 sec, 472 sec, 473 sec, 474 sec, 475 sec, 476 sec, 477 sec, 478 sec, 479 sec, 480 sec, 481 sec, 482 sec, 483 sec, 484 sec, 485 sec, 486 sec, 487 sec, 488 sec, 489 sec, 490 sec, 491 sec, 492 sec, 493 sec, 494 sec, 495 sec, 496 sec, 497 sec, 498 sec, 499 sec, 500 sec, 501 sec, 502 sec, 503 sec, 504 sec, 505 sec, 506 sec, 507 sec, 508 sec, 509 sec, 510 sec, 511 sec, 512 sec, 513 sec, 514 sec, 515 sec, 516 sec, 517 sec, 518 sec, 519 sec, 520 sec, 521 sec, 522 sec, 523 sec, 524 sec, 525 sec, 526 sec, 527 sec, 528 sec, 529 sec, 530 sec, 531 sec, 532 sec, 533 sec, 534 sec, 535 sec, 536 sec, 537 sec, 538 sec, 539 sec, 540 sec, 541 sec, 542 sec, 543 sec, 544 sec, 545 sec, 546 sec, 547 sec, 548 sec, 549 sec, 550 sec, 551 sec, 552 sec, 553 sec, 554 sec, 555 sec, 556 sec, 557 sec, 558 sec, 559 sec, 560 sec, 561 sec, 562 sec, 563 sec, 564 sec, 565 sec, 566 sec, 567 sec, 568 sec, 569 sec, 570 sec, 571 sec, 572 sec, 573 sec, 574 sec, 575 sec, 576 sec, 577 sec, 578 sec, 579 sec, 580 sec, 581 sec, 582 sec, 583 sec, 584 sec, 585 sec, 586 sec, 587 sec, 588 sec, 589 sec, 590 sec, 591 sec, 592 sec, 593 sec, 594 sec, 595 sec, 596 sec, 597 sec, 598 sec, 599 sec, and/or 600 sec.

In certain aspects, the amount or duration of the pulse is about 1 minute to 1000 minutes; or about 1 to about 100 minutes; or about 1 to about 200 minutes; or about 1 to about 300 minutes; or about 1 to about 400 minutes; or about 1 to about 500 minutes; or about 1 to about 600 minutes; or about 1 to about 700 minutes; or about 1 to about 800 minutes; or about 1 to about 900 minutes. In certain instances, the duration of treatment can be from about 1 minute to about 1000 minutes such as about 1 min, 11 min, 21 min, 31 min, 41 min, 51 min, 61 min, 71 min, 81 min, 91 min, 101 min, 111 min, 121 min, 131 min, 141 min, 151 min, 161 min, 171 min, 181 min, 191 min, 201 min, 211 min, 221 min, 231 min, 241 min, 251 min, 261 min, 271 min, 281 min, 291 min, 301 min, 311 min, 321 min, 331 min, 341 min, 351 min, 361 min, 371 min, 381 min, 391 min, 401 min, 411 min, 421 min, 431 min, 441 min, 451 min, 461 min, 471 min, 481 min, 491 min, 501 min, 511 min, 521 min, 531 min, 541 min, 551 min, 561 min, 571 min, 581 min, 591 min, 601 min, 611 min, 621 min, 631 min, 641 min, 651 min, 661 min, 671 min, 681 min, 691 min, 701 min, 711 min, 721 min, 731 min, 741 min, 751 min, 761 min, 771 min, 781 min, 791 min, 801 min, 811 min, 821 min, 831 min, 841 min, 851 min, 861 min, 871 min, 881 min, 891 min, 901 min, 911 min, 921 min, 931 min, 941 min, 951 min, 961 min, 971 min, 981 min, 991 min, and/or 1000 min.

In certain instances, the duration of treatment can be from about 1 hour to about 200 hours such as about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, 31 hrs, 32 hrs, 33 hrs, 34 hrs, 35 hrs, 36 hrs, 37 hrs, 38 hrs, 39 hrs, 40 hrs, 41 hrs, 42 hrs, 43 hrs, 44 hrs, 45 hrs, 46 hrs, 47 hrs, 48 hrs, 49 hrs, 50 hrs, 51 hrs, 52 hrs, 53 hrs, 54 hrs, 55 hrs, 56 hrs, 57 hrs, 58 hrs, 59 hrs, 60 hrs, 61 hrs, 62 hrs, 63 hrs, 64 hrs, 65 hrs, 66 hrs, 67 hrs, 68 hrs, 69 hrs, 70 hrs, 71 hrs, 72 hrs, 73 hrs, 74 hrs, 75 hrs, 76 hrs, 77 hrs, 78 hrs, 79 hrs, 80 hrs, 81 hrs, 82 hrs, 83 hrs, 84 hrs, 85 hrs, 86 hrs, 87 hrs, 88 hrs, 89 hrs, 90 hrs, 91 hrs, 92 hrs, 93 hrs, 94 hrs, 95 hrs, 96 hrs, 97 hrs, 98 hrs, 99 hrs, 100 hrs, 101 hrs, 102 hrs, 103 hrs, 104 hrs, 105 hrs, 106 hrs, 107 hrs, 108 hrs, 109 hrs, 110 hrs, 111 hrs, 112 hrs, 113 hrs, 114 hrs, 115 hrs, 116 hrs, 117 hrs, 118 hrs, 119 hrs, 120 hrs, 121 hrs, 122 hrs, 123 hrs, 124 hrs, 125 hrs, 126 hrs, 127 hrs, 128 hrs, 129 hrs, 130 hrs, 131 hrs, 132 hrs, 133 hrs, 134 hrs, 135 hrs, 136 hrs, 137 hrs, 138 hrs, 139 hrs, 140 hrs, 141 hrs, 142 hrs, 143 hrs, 144 hrs, 145 hrs, 146 hrs, 147 hrs, 148 hrs, 149 hrs, 150 hrs, 151 hrs, 152 hrs, 153 hrs, 154 hrs, 155 hrs, 156 hrs, 157 hrs, 158 hrs, 159 hrs, 160 hrs, 161 hrs, 162 hrs, 163 hrs, 164 hrs, 165 hrs, 166 hrs, 167 hrs, 168 hrs, 169 hrs, 170 hrs, 171 hrs, 172 hrs, 173 hrs, 174 hrs, 175 hrs, 176 hrs, 177 hrs, 178 hrs, 179 hrs, 180 hrs, 181 hrs, 182 hrs, 183 hrs, 184 hrs, 185 hrs, 186 hrs, 187 hrs, 188 hrs, 189 hrs, 190 hrs, 191 hrs, 192 hrs, 193 hrs, 194 hrs, 195 hrs, 196 hrs, 197 hrs, 198 hrs, 199 hrs, and/or 200 hrs.

The amount of energy of the pulse in Joules is equal to the amount of power multiplied by duration (watts×time (e.g., seconds)), so a 0.9 W/cm² applied for 9 seconds is 8.1 Joules/cm².

In one aspect, a sonosensitizer composition comprising IRDye® 700DX together with a carrier molecule is used to increase the surface area of the composition. High frequency ultrasound in the 0.1 MHz to 30 MHz range have wave lengths of about 15 mm to 0.051 mm. The large carrier molecule ensures better sonodynamic efficiencies.

In one aspect, the method of the present disclosure provides an ultrasonic wave generated from an ultrasonic transducer applied locally. In one aspect, the method of the present disclosure provides an ultrasonic wave generated from an ultrasonic transducer appended to an endoscope.

In general, ultrasonic transducers convert AC voltage into ultrasound, as well as the reverse. Ultrasonics, typically refers to piezoelectric transducers or capacitive transducers. Piezoelectric crystals change size and shape when a voltage is applied; AC voltage makes them oscillate at the same frequency and produce ultrasonic sound. Capacitive transducers use electrostatic fields between a conductive diaphragm and a backing plate. Since piezoelectric materials generate a voltage when force is applied to them, they can also work as ultrasonic detectors. Some systems use separate transmitters and receivers, while others combine both functions into a single piezoelectric transceiver. Manufacturers of commercial devices include, but are not limited to, Philips, Samsung, Siemens, Sonosite, Toshiba and Chison.

In some instances, the ultrasound device is a wearable and/or portable ultrasound device such as described in US Patent Pub. No. 2016/0136462, which is incorporated herein by reference. The disclosed wearable ultrasound device and method of using the device, which includes a power controller with a power source and at least one integrated circuit that delivers electrical power to an applicator. The applicator comprises a transducer and is electrically coupled to the power controller and a surface of the applicator transmits ultrasound to a wearer for a given duration.

Ultrasound is a mechanical wave with wavelength ranging from micrometers to centimeters. The interaction of ultrasound with bulk liquid may be accompanied by a phenomenon of cavitation that leads to concentration and conversion of sound energy. In so-called inertial cavities, gas bubbles that grow to the size of the wavelength of the sound energy can expand before collapsing, on a microscopic level. The temperature and pressure within the imploding cavities can reach such extreme levels that chemical reactions are induced within the surrounding bubble that include the generation of photons, an emission known as sonoluminescence. In addition to photons, free radicals are known to form in the cavitation bubbles that are able to react with solutes, for example, sonosensitizers, to produce products similar or the same as those formed by the interaction with light.

Different from the light excitation, ultrasound is a mechanical wave with energy that can penetrate into human body with much less of attenuation. The penetration could be multiple orders of magnitude deeper than light depending on the frequency of the application. Reactive oxygen species or singlet oxygen are generated during the ultrasound activation or sonodynamic process to introduce cytotoxicity at the treatment site. The cytoxicity can be induced by various mechanisms including apoptosis or necrosis or a combination thereof.

In certain other instances, the present disclosure provides a kit comprising a sonosensitizer composition comprising IRDye® 700DX. In certain aspects, the sonosensitizer composition is liquid or a solid such as a powder, which can be reconstituted into a liquid. The kit optionally comprises at least one syringe and/or one needle. And yet another aspect, the disclosure provides a syringe prefilled with a liquid sonosensitizer composition.

In another aspect, a kit is provided comprising a lyophilized formulation of a sonosensitizer composition comprising IRDye® 700DX and the respective amount of a liquid suitable for reconstitution. In certain embodiments, the suitable liquid is water for injection, preferable deionized sterile water for injection. In some embodiments, the kit can comprise a sonosensitizer composition comprising IRDye® 700DX and a syringe. In certain aspects, the syringe is suitable for subcutaneous injection. In other aspects, the syringe is suitable for intravenous injection. In alternative aspects, the syringe is suitable for subcutaneous injection and intravenous injection. In further aspects, the syringe is suitable for intra-arterial injection and/or intramuscular injection.

In some aspects, the kit further comprises instructions or label for use.

### III. Examples

The following examples are offered to illustrate, but not to limit, the present disclosure.

### 1. Example 1

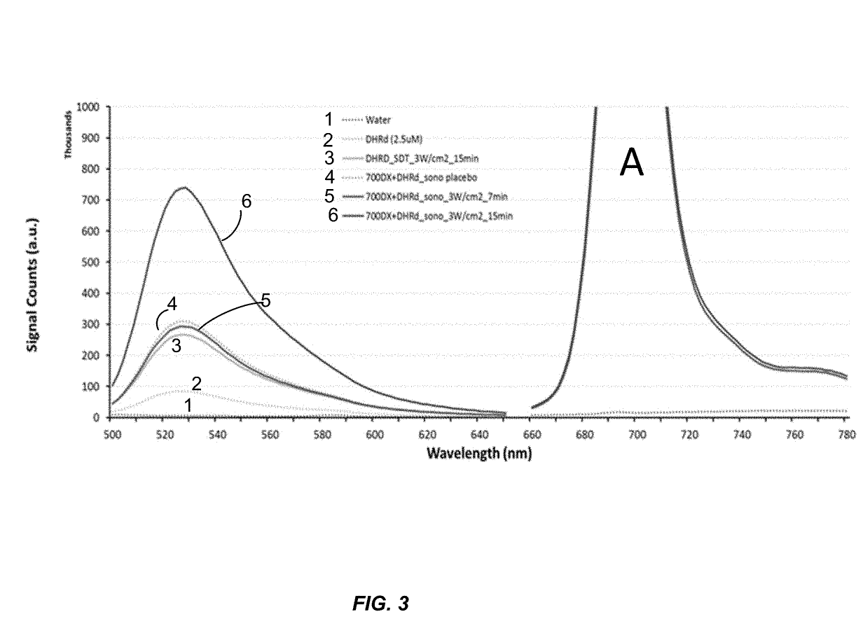

FIG. 3 shows the results of a sonodynamic experiment using methods comprising IRDye® 700DX of the present disclosure. The results illustrate that ultrasonic waves enhance the emission of a reactive oxygen species indicator, which indicates the occurrence of one of the sonodynamic effects generated by applying ultrasonic energy to IRDye® 700DX under the experimental conditions. In this example, the concentration of IRDye® 700DX was 1 μm.

The top trace (6) indicates that IRDye® 700DX is sonodynamically activated with 1MHz ultrasonic wave at 3 W/cm² for a duration of 15 minutes. A large increase in the emission of the indicator evidenced the generation of the reactive oxygen species due to the sonodymamic effect. The unaltered emission profile of IRDye® 700DX (at 700 nm range, right-hand side, “A”) implies that the chemical structure of the dye itself did not decompose in the process.

When the duration is 7 minutes (5), the emission peak is not as enhanced as with 15 minutes duration (6). The experiment included dihydroxy rhodamine 123 to detect the presence of reactive oxygen species. Reactive oxygen species were detected.

### 2. Example 2

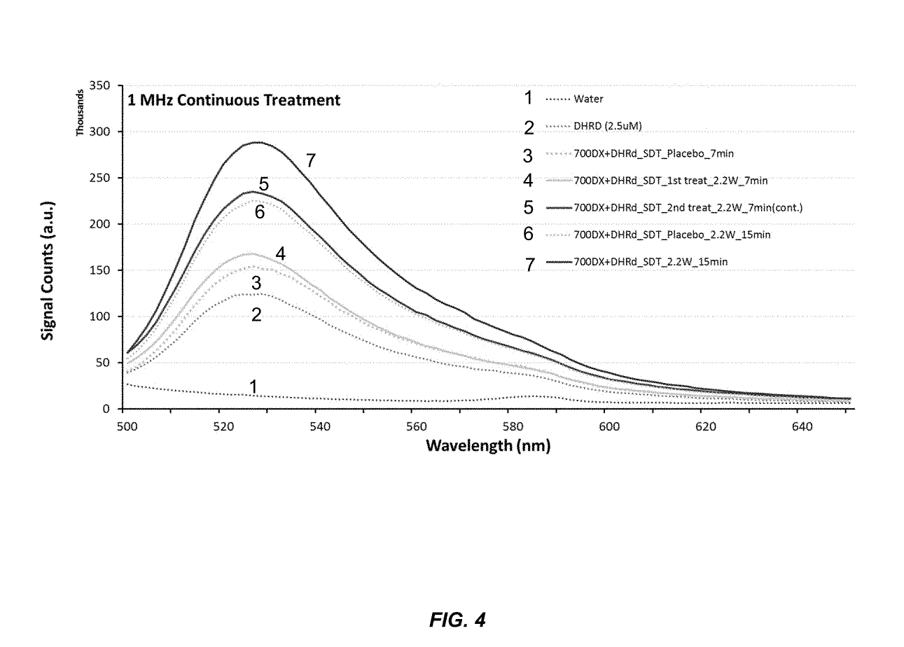

FIG. 4 shows the results of sonodynamic experiment using methods comprising IRDye® 700DX of the present disclosure. The results illustrate a continuous treatment of a 1 MHz ultrasonic wave. Increasing the pulse duration increases the generation of reactive oxygen species (compare 5 to 7). The experiment included dihydroxy rhodamine 123 to detect the presence of reactive oxygen species. Reactive oxygen species were detected.

### 3. Example 3

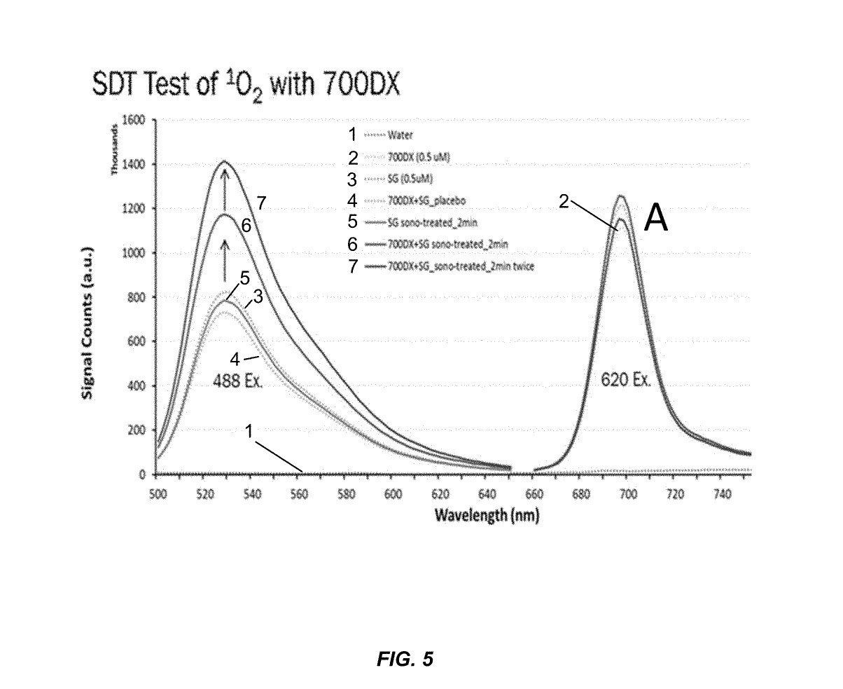

FIG. 5 shows the results of sonodynamic experiment using methods comprising IRDye® 700DX of the present disclosure. As shown therein, there is generation of singlet oxygen during the sonodynamic process. By applying the ultrasonic wave at 1MHz and 2.0W/cm² to the agent IRDye® 700DX, the enhancement of the emission of the singlet oxygen indicator, Sensor Green (SG), indicates the occurrence of a sonodynamic effects. Singlet oxygen was detected. The unaltered emission profile of IRDye® 700DX (at 700 nm range, right-hand side, “A”) implies that the structure of the dye itself was not changed in the process.

### 4. Example 4

IRDye® 700DX-labeled panitumumab is used as a sonosensitizer composition comprising IRDye® 700DX. Panitumumab is used as a targeting agent.

### 5. Example 5

A IRDye® 700DX-labeled bicelle is used as a sonosensitizer composition comprising IRDye® 700DX. Bicelles are used as carrier agents. Encapsulation of IRDye® 700DX into the lipid bilayer membrane of bicelles results in a sonosensitizer composition. A tail vein injection in a rat results in accumulation at a tumor site and thus the IRDye® 700DX-labeled bicelle acts as an efficient sonodynamic composition.

Both sonosensitization, also known as sonodynamic activation or sonoactivation, and photosensitization, also known as photodynamic activation or photoactivation, trigger reactive oxidation species (ROS) or single oxygen (¹O₂) that results in cell death via either apoptosis or necrosis, and thus sonodynamic therapy (SDT) or photodynamic therapy (PDT), respectively. To demonstrate the utility of sonoactivation for leading to SDT, various sonoactivation treatment conditions, with appropriate controls, are summarized in Table 2 below.

TABLE 2
Sonoactivation Treatment Conditions

| | | | | | | | | Total | Primary | |
|---|---|---|---|---|---|---|---|---|---|---|
| | IRDye | Sonodynamic | | | | | Duty | Energy | Sonodynamic | |
| | 700DX | Activation | Power | | Duration | | Cycle | Density | Effect Detected | |
| Water | No | No | NA | | NA | | NA | NA | NA | |
| Sensitizer DHRd123 | No | No | NA | | NA | | NA | NA | None | |
| Solution (2.5 uM) | | | | | | | | | | |
| Sensitizer DHRd123 | No | Yes | 3 | W/cm² | 15 | min | 20% | 540 J/cm² | ROS | |
| Solution | | (3.3 MHz) | | | | | | | | |
| 700DX Solution | Yes | No | 0 | | 7 | min | NA | 0 | None | With |
| (1 uM) | | | (Placebo) | | | | | | | DHRd123 |
| 700DX Solution | Yes | Yes | 3 | W/cm² | 7 | min | 20% | 252 J/cm² | ROS | With |
| | | (3.3 MHz) | | | | | | | | DHRd123 |
| 700DX Solution | Yes | Yes | 3 | W/cm² | 15 | min | 20% | 540 J/cm² | ROS++ | With |
| | | (3.3 MHz) | | | | | | | | DHRd123 |
| 700DX Solution | Yes | No | 0 | | 7 | min | NA | 0 | None | With |
| (1 uM) | | | (Placebo) | | | | | | | DHRd123 |
| 700DX Solution | Yes | Yes | 2.2 | W/cm² | 7 | min | 20% | 185 J/cm² | ROS | With |
| | | (1 MHz) | | | | | | | | DHRd123 |
| 700DX Solution | Yes | Yes | 2.2 | W/cm² | 7 + 7 | min | 20% | 370 J/cm² | ROS+ | With |
| | | (1 MHz) | | | | | | | | DHRd123 |
| 700DX Solution | Yes | No | 0 | | 15 | min | NA | 0 | Minimal | With |
| (1 uM) | | | (Placebo) | | | | | | effect | DHRd123 |
| | | | | | | | | | observed | |
| 700DX Solution | Yes | Yes | 2.2 | W/cm² | 15 | min | 20% | 396 J/cm² | ROS++ | With |
| | | (1 MHz) | | | | | | | | DHRd123 |
| Sensor Green | No | No | NA | | NA | | NA | NA | NA | |
| (0.5 uM) | | | | | | | | | | |
| Sensor Green | No | Yes | 2 | W/cm² | 2 | min | 50% | 120 J/cm² | None | |
| | | (1 MHz) | | | | | | | | |
| 700DX Solution | Yes | Yes | 2 | W/cm² | 2 | min | 50% | 120 J/cm² | ¹O₂+ | With |
| | | (1 MHz) | | | | | | | | Sensor Green |
| 700DX Solution | Yes | Yes | 2 | W/cm² | 2 + 2 | min | 50% | 240 J/cm² | ¹O₂++ | With |
| | | (1 MHz) | | | | | | | | Sensor Green |

The formation of ROS or ¹O₂ as confirmed by DHRd123 (dihydrorhodamine 123) or Sensor Green, respectively, by either sonoactivation or photoactivation leads to cell death as described in Examples 6 and 7 below. Although Examples 6 and 7 reflect the formation of ROS or ¹O₂ via photoactivation, it is to be understood by one skilled in the art that the formation of ROS or ¹O₂ via sonoactivation would have an equivalent cell death result.

### 6. Example 6

This example illustrates the use of IRDye® 700Dx small molecule conjugates (probes) to induce programmed cell death, i.e., apoptosis in cells. The small molecule conjugates described herein include IRDye® 700DX CLTX (chlorotoxin) and IRDye® 700DX anti-EGFR Affibody®.

Chlorotoxin (CLTX) is a 36 amino acid peptide found in venom of Leiurus quinquestriatu. It can block small-conductance chlorine channels. The molecule has also been shown to bind annexin A2 receptors and has a dual effect on the enzymatic activity of MMP-2.

Affibody® (Affibody, Solna Sweden) affinity ligands are described as antibody mimetics with superior characteristics. They are approximately 6 kDa in size and no Fc function. Affibodies also incorporate the Alburnod™ technology that extends their circulatory half-life through a strong binding to albumin. Commercially available Affibody® molecules include anti-EGFR Affibody®, anti-ErbB2 Affibody®, anti-fibrinogen Affibody®, anti-insulin Affibody®, anti-TNFα Affibody®, anti-Affibody®, etc.

A. IRDye® 700DX-labeled CLTX

The procedure of labeling CLTX with IRDye® 700Dx is similar to that of labeling CLTX with IRDye® 800CW (Kovar et al., Anal i, 2013, 440(2):212-9). The basic structure was not altered and binding occurs with exposed lysine residues. The D/P ratio was ˜2. Dilutions were made and run on a bis-Tris glycine gel for visualization. The overall size of the labeled molecule was estimated to be about 5950 MW.

For the treatment study, HTB-186 cells (a desmoplastic cerebellar medulloblastoma cell line) were prepared and plated in petri dishes. The cells were incubated overnight at 37° C., 5% CO₂. Treatments with respective probe and irradiation levels are presented in the Table. The treatment dose was 10 μl per 500 μl plate. The probes were incubated on the cells for about 5 hours at 37° C., 5% CO₂.
Treatment Conditions

TABLE 3

| # | Probe | Irradiation |
|---|---|---|
| 1 | Control: No probe | No IRR |
| 2 | Control: No probe | 32 J/cm² |
| 3 | CLTX-700DX (0.2 μg/ml) | No IRR |
| 4 | CLTX-700DX (0.2 μg/ml) | 16 J/cm² |
| 5 | CLTX-700DX (0.2 μg/ml) | 32 J/cm² |

At several time points post irradiation, cells were evaluated for morphology changes using epi-fluorescent microscope. Treatments were imaged after incubation with the probe and before irradiation to document morphology. In addition, cells were images at the following timepoints: immediately after, 1 h, 2 h, and 24 h post irradiation.

Immediately after incubation and prior to irradiation, the cells from all the treatments (treatments 1-5) were healthy in appearance. No change in cell morphology was detected in any of the treatments. Those treatments receiving probe (treatments 3-5) showed similar punctate incorporation of the probe into the cell. 700 nm images showed the probe was internalized into the cells by endocytosis.

Immediately after irradiation, the cell morphology still remained healthy for all treatments including those receiving irradiation (treatment 2, 4 and 5). Signals captured in the 700 nm channel show that the probe intensity was similar among cells receiving probe (treatments 3, 4, and 5).

At 2h post irradiation the punctate pattern of the probe remained unchanged from the initial images for the treatment receiving no irradiation (treatment 3). However, a change was detected for treatment 5 which received the highest level of irradiation. The punctate pattern appeared brighter and more intracellularly located. The morphology of the cells of treatment 5 remained normal compared to the other treatments.

At 24 hours post irradiation, the discernible changes in the 700 nm signal noted at 2 h appears were more pronounced. The bright signals from the probe were now localized inside the cells and in some cases, in particular regions of the cells. In addition, the characteristic blebbing and rounded appearance indicative of cells undergoing apoptosis was also detected.

The appearance of blebbing of the cells after more than 2 hours post irradiation suggests that the cells are undergoing apoptosis, and not necrosis which occurs on a faster time frame. For example, a necrotic response to an photodynamic antibody probe occurs within 15 minutes post irradiation.

To further investigate the subcellular localization of the IRDye® 700Dx small molecule probe, we used fluorescent organelle specific dyes to look for colocalization. We used MitoTracker® Green specific to the mitochondria to determine if the internalization of CLTX-700DX placed the probe at the mitochondrial. HTB-186 cells were plated on glass coverslips in petri-dishes and allowed to equilibrate for 24 h in complete media. The cells were incubated with CLTX-700DX for 4-5 h after which the cells were irradiated at 32 J/cm². Plates were incubated for an additional 24 h at 37° C. 5% CO₂. Cells were treated for 45 min with MitoTracker® Green per manufacturer's instruction. Cells were gently rinsed and incubated for 15 min with DAPI to stain the nuclei. Additional rinses were performed and the coverslip mounted on glass slides with Fluoromount™ medium. Microscopy imaging was performed to document location of the fluorophores and probe.

Microscopy analysis reveals that the CLTX-700DX probe was not located in the mitochondria or nuclei. The punctate pattern of the probe visualized 4-5 hours after incubation shows that the probe is internalized by the cell by endocytosis.

B. IRDye® 700DX-labeled anti-EGFR Affibody®

The anti-EGFR Affibody® was conjugated with IRDye® 700DX via the specific cysteine residue engineered on the Affibody®. IRDye® 700DX maleimide was prepared and effectively used to label at a D/P of 1.

For the treatment study, A431 cells (an epidermoid carcinoma cell line) were seeded on a coverslip in a petri-dish and incubated for 24 h. The cells were incubated overnight at 37° C., 5% CO₂. Treatments with respective probes and irradiation levels are presented in the Table. The treatment dose was 10 μl per 500 μl plate. The probes were incubated on the cells for about 4-5 hours at 37° C., 5% CO₂. Cells were rinsed and irradiated at levels shown above. Petri-dishes were placed back in the incubator for up to 24 h.
Treatment Conditions

TABLE 4

| # | Probe | Irradiation |
|---|---|---|
| 1 | Control: No probe | No IRR |
| 2 | Control: No probe | 32 J/cm² |
| 3 | 700DX-EGFR Affibody ® | No IRR |
| 4 | 700DX-EGFR Affibody ® | 16 J/cm² |
| 5 | 700DX-EGFR Affibody ® | 32 J/cm² |

Images were captured before irradiation and then at 1 h, 2 h, and 24 h after irradiation to follow any morphology changes indicative of apoptosis or necrosis.

Initial imaging before irradiation showed very healthy cell growth with good labeling of the cell membrane by IRDye® 700DX anti-EGFR Affibody® in treatments 3-5.

No morphology changes were detected for the control conditions without probe (treatments 1 and 2). At 1 h post irradiation cell rounding and shrinkage was visible in cells of treatment 4, and to a greater extent in cells of treatment 5. The cell surface location of the probe in cells of treatment 3 was different compared to the localization in cells of treatment 4 and 5. In these two treatments, the probe was located in more discrete locations of the cell. The localization of the probe may be due directly or indirectly to the altered cell shape or as a consequence of apoptosis.

At 2 h post irradiation, the cells of treatments 4 and 5 remain rounded and beginning to exhibit blebbing which is a hallmark of apoptosis. The cells of treatment 5 also appear less healthy compared to early time points and to the cells of the other treatments.

At 24 h post irradiation, treatments 1, 2, and 3 all appear normal in appearance. No effect of irradiation (32 J/cm²) was detected in cells that did not receive the probe (treatment 2). Also, no effect of probe on cells without irradiation was detected (treatment 3).

The cells of treatment 4 appear similar in appearance and morphology as at the early time point of 2 h post irradiation. The data suggests that the cells initiated the programmed cell death pathway and did not progress to late apoptosis. Some cells appeared to have normal, healthy morphology, which suggests that the treatment conditions of treatment 4 are sub-lethal.

The cells of treatment 5 which received a higher level of irradiation (32 J/cm²) exhibited complete cell disruption and a dramatic change in morphology that appears similar to that seen in cells treated with IRDye® 700DX labeled antibodies. No healthy cells were observed with these treatments. It should be noted that it takes IRDye® 700DX labeled small molecule probes longer (more time) to kill cells than IRDye® 700DX labeled antibodies. Since apoptosis occurs over a longer time period, it appears that IRDye® 700DX labeled small molecule probes induce apoptosis and IRDye® 700DX labeled antibodies induce necrosis.

To further investigate the subcellular localization of the IRDye® 700DX anti-EGFR Affibody®, we used MitoTracker® Green as described above. A431 cells were plated on glass coverslips in petri-dishes and allowed to equilibrate for 24 h in complete media. The cells were incubated with IRDye® 700DX anti-EGFR Affibody® for 4-5 h, after which cells were irradiated (32 J/cm²). Plates were incubated for an additional 24 h at 37° C. 5% CO₂. Cells were treated for 45 min with MitoTracker® Green per manufacturer's instruction. Cells were gently rinsed and incubated for 15 min with DAPI to stain nuclei. Additional rinses were done and the coverslip mounted on glass slides with Fluoromount™ medium. The fluorophores and probe were detected by epi-fluorescence microscopy. The IRDye® 700DX anti-EGFR Affibody® was not located in the mitochondria or the nuclei.

In summary, the example provided herein illustrates the use of IRDye® 700DX labeled small molecule probes for photodymamic therapy. The data shows that the probes are internalized (endocytosed) by the cells, and upon exposure to irradiating light, the cells undergo apoptosis.

### 7. Example 7

This example shows an evaluation of various targeting agents and IRDye® 700DX NHS ester labeling of cells. A small molecule RGD labeled probe was tested. It should be noted that A431 cells express very low levels of integrin receptors and are not the ideal cell type for this probe.

Briefly, A431 cells were labeled with respective probes for specific treatments (listed below); cells were rinsed and evaluated for labeling. The test include the following treatments: treatments 1-2 of RGD-IRDye® 700DX at 1 μM and radiation, treatments 3-4 of EGF-IRDye® 700DX at 0.5 μM and radiation, treatments 5-6 of IRDye® 700DX NHSe at 5 μM and radiation, treatments 7-8 of panitumumab-IRDye® 700DX at about 0.1 μM and radiation, treatment 9 of a negative control with no probe and no irradiation, and treatment 10 of RGD-IRDye® 700DX and no irradiation. The probes were incubated for about 10 minutes and with a longer period of about 20 minutes for the RGD probe. The cells were monitored with microscopy and morphology was examination 24 hours post treatment.
Table % vitality, viability and necrosis of treatments 1-10.

TABLE 5

| | (32 | | Est | | Low | % |
|---|---|---|---|---|---|---|
| | J/cm²) | | % Vi- | % | % Vi- | Ne- |
| VB-48 Assay | Irradi- | Cells/ | abil- | Heal- | tal- | cro- |
| Treatment | ation | mL | ity | thy | ity | tic |
| 1-2 RGD-700DX | I | 6.6 × 10⁵ | 90-100 | 91.6 | 2 | 7 |
| (0.5 μM) | | | | | | |
| 3-4 EGF-700DX | I | 3.4 × 10⁵ | 60-70 | 60 | 19 | 20 |
| (0.5 μM) | | | | | | |
| 5-6 700DX NHSe | I | 1.8 × 10⁵ | 20-30 | 2.5 | 25 | 72 |
| (5 μM) | | | | | | |
| 7-8 Pan-700DX | I | 1.2 × 10⁵ | 30-40 | 15.0 | 15 | 70 |
| (~0.1 μM) | | | | | | |
| 9 Control | NI | 6.4 × 10⁵ | 90-100 | 94.8 | 3 | 2 |
| 10 RGD-700DX | NI | 5.8 × 10⁵ | 90-100 | 90.8 | 4 | 5 |
| (0.5 μM) | | | | | | |

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Informal Sequence Listing

Sequences
Variant HPV16/3 1 LI protein nucleotide sequence
(SEQ ID NO: 1)
ATGAGCCTGTGGCTGCCCAGCGAGGCCACCGTGTACCTGCCCCCCGTGCC
CGTGAGCAAGGTGTGAGCACCGACGAGTACGTGGCCAGGACCAACATCTA
CTACCACGCCGGCACCAGCAGGCTGCTGGCCGTGGGCCACCCCTACTTCC
CCATCAAGAAGCCCAACAACAACAAGATCCTGGTGCCCAAGGTGAGCGGC
CTGCAGTACAGGGTGTTCAGGATCCACCTGCCCGACCCCAACAAGTTCGG
CTTCCCCGACACCAGCTTCTACAACCCCGACACCCAGAGGCTGGTGTGGG
CCTGCGTGGGCGTGGAGGTGGGCAGGGGCCAGCCCCTGGGCGTGGGCATC
AGCGGCCACCCCCTGCTGAACAAGCTGGACGACACCGAGAACGCCAGCGC
CTACGCCGCCAACGCCGGCGTGGACAACAGGGAGTGCATCAGCATGGACT
ACAAGCAGACCCAGCTGTGCCTGATCGGCTGCAAGCCCCCCATCGGCGAG
CACTGGGGCAAGGGCAGCCCCTGCACCAACGTGGCCGTGAACCCCGGCGA
CTGCCCCCCCCTGGAGCTGATCAACACCGTGATCCAGGACGGCGACATGG
TGGACACCGGCTTCGGCG
CCATGGACTTCACCACCCTGCAGGCCAACAAGAGCGAGGTGCCCCTGGAC
ATCTGCACCAGCATCTGCAAGTACCCCGACTACATCAAGATGGTGAGCGA
GCCCTACGGCGACAGCCTGTTCTTCTACCTGAGGAGGGAGCAGATGTTCG
TGAGGCACCTGTTCAACAGGGCCGGCGCCGTGGGCGAGAACGTGCCCACC
GACCTGTACATCAAGGGCAGCGGCAGCACCGCCACCCTGGCCAACAGCAA
CTACTTCCCCACCCCCAGCGGCAGCATGGTGACCAGCGACGCCCAGATCT
TCAACAAGCCCTACTGGCTGCAGAGGGCCCAGGGCCACAACAACGGCATC
TGCTGGGGCAACCAGCTGTTCGTGACCGTGGTGGACACCACCAGGAGCAC
CAACATGAGCCTGTGCGCCGCCATCAGCACCAGCGAGACCACCTACAAGA
ACACCAACTTCAAGGAGTACCTGAGGCACGGCGAGGAGTACGACCTGCAG
TTCATCTTCCAGCTGTGCAAGATCACCCTGACCGCCGACGTGATGACCTA
CATCCACAGCATGAACAGCACCATCCTGGAGGACTGGAACTTCGGCCTGC
AGCCCCCCCCCGGCGGCACCCTGGAGGACACCTACAGGTTCGTGACCAGC
CAGGCCATCGCCTGCCAGAAGCACACCCCCCCCGCCCCCAAGGAGGACCC
CCTGAAGAAGTACACCTTCTGGGAGGTGAACCTGAAGGAGAAGTTCAGCG
CCGACCTGGACCAGTTCCCCCTGGGCAGGAAGTTCCTGCTGCAGGCCGGC
CTGAAGGCCAAGCCCAAGTTCACCCTGGGCAAGAGGAAGGCCACCCCCAC
CACCAGCAGCACCAGCACCACCGCCAAGAGGAAGAAGAGGAAGCTGTGA
BPV1 LI nucleotide sequence (SEQ ID NO: 2)
ATGGCCCTCTGGCAGCAGGGGCAGAAACTCTACCTGCCACCCACACCCGT
GTCAAAAGTCCTGTGTTCCGAGACATACGTCCAGCGGAAGTCAATCTTCT
ACCACGCCGAGACCGAAAGGCTCCTCACCATCGGCCACCCCTACTACCCC
GTCAGCATTGGCGCTAAGACCGTGCCCAAAGTCTCCGCCAACCAATACCG
CGTGTTCAAGATCCAGCTGCCCGACCCAAACCAGTTCGCCCTGCCCGATC
GCACCGTGCATAACCCCTCCAAGGAAAGACTCGTCTGGGCCGTGATCGGC
GTCCAAGTCTCACGGGGCCAACCCCTGGGCGGCACCGTGACCGGCCATCC
AACCTTCAACGCCCTCCTGGACGCCGAGAACGTCAACCGGAAAGTCACAA
CACAAACCACCGACGATCGCAAGCAGACCGGGCTGGACGCCAAACAGCAG
CAAATCCTCCTCCTGGGGTGCACACCCGCTGAGGGCGAGTACTGGACCAC
CGCTCGGCCCTGCGTGACCGACAGGCTGGAGAACGGGGCTTGTCCCCCCC
TGGAGCTGAAGAATAAGCATATCGAGGACGGCGACATGATGGAGATCGGC
TTCGGCGCCGCTAACTTCAAGGAGATCAACGCCTCCAAGAGCGACCTGCC
CCTGGATATCCAGAACGAAATTTGTCTCTATCCCGATTATCTGAAGATGG
CCGAAGATGCCGCCGGCAACTCAATGTTTTTCTTCGCCCGCAAGGAGCAA
GTCTACGTGCGGCATATTTGGACACGGGGCGGGAGCGAAAAGGAGGCTCC
CACAACCGACTTCTACCTGAAAAACAACAAGGGCGACGCTACACTGAAGA
TCCCATCCGTCCACTTCGGCTCCCCATCCGGGAGCCTCGTCAGCACCGAC
AACCAGATCTTCAACAGACCATATTGGCTGTTTAGGGCTCAAGGGATGAA
TAACGGCATCGCTTGGAACAACCTGCTCTTCCTGACCGTCGGCGATAACA
CCAGGGGCACCAACCTGACAATCTCCGTGGCTAGCGACGGCACACCCCTG
ACCGAATACGACTCAAGCAAGTTTAACGTGTATCACCGGCACATGGAGGA
GTACAAACTGGCTTTCATCCTGGAACTGTGTAGCGTCGAGATTACCGCCC
AGACCGTCAGCCACCTCCAGGGCCTGATGCCAAGCGTCCTGGAGAACTGG
GAGATCGGCGTCCAACCACCAACAAGCAGCATCCTGGAAGATACATACAG
ATACATCGAAAGCCCCGCCACCAAGTGCGCCTCAAACGTGATCCCCGCCA
AGGAGGATCCCTACGCCGGCTTCAAATTCTGGAATATCGACCTGAAGGAG
AAACTGAGCCTCGATCTGGACCAGTTCCCACTCGGCCGGCGGTTCCTGGC
CCAACAGGGCGCTGGCTGCAGCACCGTCCGGAAGAGGCGGATCTCACAAA
AGACCAGTTCCAAACCCGCCAAGAAGAAGAAGAAGTAG

What is claimed is:

1. A method for treating a diseased cell in a subject using sonodynamic therapy, said method comprising:
administering to the subject a sonosensitizer composition comprising IRDye® 700DX, wherein the sonosensitizer associates with the diseased cell; and
applying an ultrasonic wave to the diseased cell.

2. The method of claim 1, wherein said diseased cell is a cancer cell.

3. The method of claim 2, wherein said cancer cell is a cell of a solid tumor.

4. The method of claim 3, wherein said solid tumor is a member selected from the group consisting of lung, breast, bladder, ovarian, pancreatic, skin, esophagus, stomach, liver, colon and prostate cancer.

5. The method of claim 1, wherein said sonosensitizer composition comprises a carrier moiety.

6. The method of claim 5, wherein said carrier moiety is a virus-like particle.

7. The method of claim 6, wherein said virus-like particle comprises a L1 caspid protein, a L2 caspid protein or a combination thereof

8. The method of claim 5, wherein said carrier moiety is a nanotube.

9. The method of claim 1, wherein said sonosensitizer composition comprises a targeting agent.

10. The method of claim 9, wherein said targeting agent is an antibody.

11. The method of claim 1, wherein said administration comprises systemic administration to the subject, local administration to a tumor, or administration to a surgical site.

12. The method of claim 1, wherein said ultrasonic wave is applied at a frequency of about 0.1 MHz to about 30 MHz.

13. The method of claim 12, wherein said ultrasonic wave is applied at a frequency of about 1.0 MHz to about 5.0 MHz.

14. The method of claim 12, wherein said ultrasonic wave is applied at a frequency of about 1.0 MHz to about 2.0 MHz.

15. The method of claim 1, wherein said ultrasonic wave is applied at a power density of about 0.01 W/cm² to about 12 W/cm².

16. The method of claim 15, wherein said ultrasonic wave is applied at a power density of about 1.0 W/cm² to about 6 W/cm².

17. The method of claim 15, wherein said ultrasonic wave is applied at a power density of about 1.2 W/cm² to about 3.8 W/cm².

18. The method of claim 1, wherein the ultrasonic wave is generated from an ultrasonic transducer applied locally.

19. The method of claim 1, wherein the ultrasonic transducer is appended to an endoscope.

20. A kit, the kit comprising a sonosensitizer composition comprising IRDye® 700DX.
